(19) United States
(12) Patent Application Publication
(10) Pub. No.: US 2021/0280296 A1
(43) Pub. Date: Sep. 9, 2021

(54) INTERACTIVE SYSTEM FOR IMPROVED MENTAL HEALTH

(71) Applicant: Integrated Mental Health Technologies, LLC, Centennial, CO (US)

(72) Inventor: Sarah Houy, Centennial, CO (US)

(73) Assignee: Integrated Mental Health Technologies, LLC, Centennial, CO (US)

(21) Appl. No.: 17/193,190

(22) Filed: Mar. 5, 2021

Related U.S. Application Data
(60) Provisional application No. 62/986,613, filed on Mar. 6, 2020.

(51) Int. Cl. G16H 20/70; G16H 10/20

(52) U.S. Cl. CPC G16H 20/70; G16H 10/20

(57) ABSTRACT

Systems and methods for improved mental health. A mental health system includes an interactive awareness and support tool. In one aspect, the interactive awareness and support tool generates custom guided exercises, insights, and reminders based on user-provided inputs, experiences, observations and/or self-reflection. User inputs are facilitated through a tagging system operating on aggregated and synthesized user-provided data to construct a user mental health database which may be filtered and leveraged by a user to increase mental health and enable on-demand mental health therapy. The mental health database enables construction and rendering of mental health mappings, such as mappings of sub-personalities with respect to Self.

### CROSS-REFERENCE TO RELATED APPLICATION

This application is a nonprovisional patent application of and claims the benefit of U.S. Provisional Patent Application No. 62/986,613 filed Mar. 6, 2020 and titled “Interactive System for Improved Mental Health,” the disclosure of which is hereby incorporated herein by reference in entirety.

### FIELD

The disclosure relates generally to systems and methods for mental health, and more specifically to an interactive system and associated method for improved mental health.

### BACKGROUND

Mental health at a very basic level is our ability to maintain self-regulation and includes our emotional, psychological and social well-being which results in overall well-being. When our mental health is struggling often this is referred to as emotional distress.

Psychological modalities are proven methods that help individuals deal with this ebb & flow of emotional, psychological and social well-being though understanding, uncovering and connecting individuals to inner and outer resources. There are a multitude of psychological modalities including Cognitive Behavior Therapy (CBT), Prolonged Exposure (PE), Eye Movement Desensitization and Reprocessing (EMDR), and Internal Family Systems Therapy (IFS), for example. Internal Family Systems therapy is an evidence-based method of psychotherapy which helps individuals harmonize the mind and increase overall well-being. Even though technology has not played a large role in the field of psychology in the past it is rapidly showing growth as more individuals see the benefit of responsibly integrating technology to improve their overall well-being. This has been done for some time with the physical well-being aspect with apps that help track exercise and nutrition. What is needed is an awareness and support tool that responsibly leverages technology and proper deep integration with evidence-based psychotherapy methods to assist in the psychological, emotional and social well-being of individuals. The disclosure solves this need. The benefits of the disclosed awareness and support tool to a client user with mental health challenges include new and enriched awareness, new and enriched support, and decreased barriers to change, which leads to overall decreased emotional distress and increased emotional, psychological and social well-being.

### SUMMARY

A system and method of use for mental health, and more specifically a system and method for interactive mental health is disclosed. (In the three embodiments summarized below in this Summary section, the term “trigger” is as described in e.g., FIGS. 2B, 3A, and 3B, and is akin to the term “trailhead” as described in e.g., FIGS. 8B, 14A, and 14B. The term “parts” is as described in e.g., FIG. 8B and 15A-H and 15J and is akin to the term “States” as described in e.g., FIGS. 3A and 3C).

In one embodiment, and awareness and support tool to improve user well-being of a user is disclosed, the tool comprising: a user interface operating to receive temporally-marked user data sets comprising: i) user state data comprising user mental state and user physical state; ii) user trigger data; iii) user self-assessment data; and iv) user self-reflection data; a user database operating to: i) store the temporally-marked user data sets; ii) sort the temporally-marked user data sets; and iii) filter the temporally-marked user data sets; a system database operating to store a set of guided exercises associated with at least user state data, user trigger data, and user self-assessment data; a processor operating on the user database to determine mappings between data of the temporally-marked user data sets and to identify insights associated with data of the temporally-marked user data sets; wherein: the user inputs, through the user interface, a first set of user self-assessment data and a first set of user state data; the processor selects, based on the first set of user self-assessment data and the first set of user state data, a first guided exercise from the system database, and renders the first guided exercise on the user interface; the user executes the guided exercise and inputs, through the user interface, a first set of user self-reflection data associated with executing the first guided exercise; the user inputs, through the user interface, a first set of user trigger data; the processor maps the first set of user state data and the first set of user trigger data with respect to a set of pre-existing temporally-marked user data sets to create a first visual map; the processor renders, on the user interface, the first visual map; the processor generates a first insight associated with the first set of user state data, the first set of user trigger data, and the set of pre-existing temporally-marked user data; the processor renders the first insight; and the user inputs, through the user interface, a second set of user self-assessment data.

In one aspect, the processor compares the first set of user self-assessment data and the second set of user self-assessment data to generate a second insight, the second insight render on the user interface by the processor. In another aspect, the first set of user self-assessment data comprises a visual sliding scale data entry. In another aspect, the first visual map comprises a particular set of user state data for a selected user trigger data. In another aspect, the set of user state data is rendered as a first particular set of user state data and a second particular set of user data. In another aspect, the user enters a time reminder to view a selected trigger. In another aspect, the temporally-marked user data sets further comprise user parts data. In another aspect, the processor renders, on the user interface, a second visual map comprising a particular set of user state data for a selected user parts data. In another aspect, the processor and user interface reside on a portable computer device. In another aspect, at least part of the user database and the system database reside external to the portable computer device.

In another embodiment, an awareness and support tool to improve user well-being of a user is disclosed, the tool comprising: a user database operating to i) store a plurality of user part data sets, each user part data set comprising a set of current user state data and a set of existing user state data; ii) sort the plurality of user part data sets with respect to a particular set of user state data; and iii) filter the plurality of user part data sets with respect to the particular set of user state data; a user interface operating to receive: i) the set of current user state data, the current user state data comprising user mental state and user physical state; and ii) user self-assessment data; a system database operating to store a set of guided exercises associated with at least the current user state data; a processor operating on the user database to determine mappings between the plurality of user part data sets and at least one of the set of current user state data and the set of existing user state data; wherein: the processor generates and renders a first visual mapping of a relationship between the plurality of user part data sets and at least one of the set of current user state data and the set of existing user state data; the user selects the particular set of user state data as rendered on the user interface by the processor; the user enters the set of current user state data associated with the particular set of user state data; and the processor selects, based on the set of current user state data, a first guided exercise from the system database, and renders the first guided exercise on the user interface.

In one aspect, the user executes the guided exercise and inputs, through the user interface, a first set of user self-reflection data associated with executing the first guided exercise. In another aspect, the first set of user self-assessment data comprises a visual sliding scale data entry. In another aspect, the processor generates a first insight associated with the set of current user state data. In another aspect, the user inputs, through the user interface, i) a first set of user self-assessment data before the user selects the particular set of user state data, and ii) a second set of user self-assessment data after the user executes the first guided exercise.

In another embodiment, a method of improving user well-being of a user is disclosed, the method comprising: providing an awareness and support tool comprising: to a user interface operating to receive temporally-marked user data sets comprising: i) user state data comprising user mental state and user physical state; ii) user trigger data; iii) user self-assessment data; and iv) user self-reflection data; a user database operating to: i) store the temporally-marked user data sets; ii) sort the temporally-marked user data sets; and iii) filter the temporally-marked user data sets; a system database operating to store a set of guided exercises associated with at least user state data, user trigger data, and user self-assessment data; and a processor operating on the user database to determine mappings between data of the temporally-marked user data sets and to identify insights associated with data of the temporally-marked user data sets; inputting, by the user through the under interface, a first set of user self-assessment data and a first set of user state data; selecting, by the processor and based on the first set of user self-assessment data and the first set of user state data, a first guided exercise from the system database; rendering, by the processor, the first guided exercise on the user interface; executing, by the user, the guided exercise; inputting, by the user through the user interface, a first set of user self-reflection data associated with executing the first guided exercise; inputting, through the user interface, a first set of user trigger data; mapping, by the processor, the first set of user state data and the first set of user trigger data with respect to a set of pre-existing temporally-marked user data sets to create a first visual map; rendering, by the processor on the user interface, the first visual map; generating, by the processor, a first insight associated with the first set of user state data, the first set of user trigger data, and the set of pre-existing temporally-marked user data; rendering, by the processor, the first insight; and inputting, by the user through the user interface, a second set of user self-assessment data.

In one aspect, the first set of user self-assessment data comprises a visual sliding scale data entry. In another aspect, the first visual map comprises a particular set of user state data for a selected user trigger data. In another aspect, the set of user state data is rendered as a first particular set of user state data and a second particular set of user data. In another aspect: i) the processor and user interface reside on a portable computer device; and ii) at least part of the user database and the system database reside external to the portable computer device.

By way of providing additional background, context, and to further satisfy the written description requirements of 35 U.S.C. § 112, the following references are incorporated by reference in their entireties: US Pat. Appl. Nos. 2018/0308574 to Beauchamp and 2016/0378928 to Benton.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably. The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The terms “determine”, “calculate” and “compute,” and variations thereof, as used herein, are used interchangeably and include any type of methodology, process, mathematical operation or technique.

The term “means” as used herein shall be given its broadest possible interpretation in accordance with 35 U. S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “app” means a software application typically accessed by way of an icon displayed on a user interface of a computer, such as a portable computer.

The term “part/s” may be used interchangeably with the term “state/s,” such as when the IFS psychotherapy model 104 is employed.

The term “trailhead” may be used interchangeably with the term “trigger,” such as when the IFS psychotherapy model 104 is employed

The term “Self” may be used interchangeably with the term “core self,” such as when the IFS psychotherapy model 104 is employed.

Various embodiments or portions of methods of manufacture may also or alternatively be implemented partially in software and/or firmware, e.g., analysis of signs. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, firmware code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below. Also, while the disclosure is presented in terms of exemplary embodiments, it should be appreciated that individual aspects of the disclosure can be separately claimed.

### BRIEF DESCRIPTION OF THE DRAWINGS

The disclosure will be readily understood by the following detailed description in conjunction with the accompanying drawings, wherein like reference numerals designate like elements. The elements of the drawings are not necessarily to scale relative to each other. Identical reference numerals have been used, where possible, to designate identical features that are common to the figures.

FIG. 1 is a schematic representation of one embodiment of an interactive support and awareness tool to improve user well-being;

FIG. 2A is a block diagram representation of one embodiment of the Workflow core module element of the interactive support and awareness tool and associated method for improved mental health FIG. 1A;

FIG. 2B is a flow diagram of a method of use of the Workflow core module element of FIG. 2A;

FIG. 3A is a block diagram representation of one embodiment of the observations core module element of the interactive support and awareness tool of FIG. 1A;

FIG. 3B is a flow diagram of a method of use of the triggers submodule of the observations core module element of FIG. 2A;

FIG. 3C is a flow diagram of a method of use of the states submodule of the observations core module element of FIG. 2A;

FIG. 4A is a block diagram representation of one embodiment of the AI insights core module element of the embodiment of the interactive support and awareness tool FIG. 1A;

FIG. 4B is a flow diagram of a method of use of the user insight core module element of FIG. 4A;

FIG. 5 is a block diagram representation of one embodiment of the user insight core module element of the interactive support and awareness tool of FIG. 1A;

FIG. 6 is a block diagram representation of one embodiment of the Customized Content core module of the interactive support and awareness tool of FIG. 1A;

FIG. 7 is a block diagram representation of one embodiment of the Professional Support, guided exercises, and User Community core modules of the interactive support and awareness tool of FIG. 1A;

FIG. 8A is one embodiment of an upper portion of a user interface home screen of the interactive support and awareness tool of FIG. 1A (FIG. 8B provides the companion lower portion);

FIG. 8A is one embodiment of a lower portion of a user interface home screen of the interactive support and awareness tool of FIG. 1A (FIG. 8A provides the companion upper portion);

FIG. 9A is one embodiment of a user interface to receive user state data via a tagging system of the interactive support and awareness tool of FIG. 1A;

FIG. 9B is one embodiment of a user interface to receive user state data via a tagging system accessed by way of the user interface of FIG. 9A;

FIG. 10A is one embodiment of a user interface to select custom guided exercises of the interactive support and awareness tool h of FIG. 1A;

FIG. 10B is another embodiment of a user interface to select user observations via the tagging system from the custom guided exercises of FIG. 10A;

FIG. 11A is one embodiment of a user interface to categorize a trigger of the interactive support and awareness tool of FIG. 1A;

FIG. 11B is another embodiment of a user interface to set a reminder to check back in with a trigger of the interactive support and awareness tool of FIG. 1A;

FIG. 12A is one embodiment of a user interface with a support bot of the interactive support and awareness tool of FIG. 1A;

FIG. 12B is one embodiment of a user interface to select additional custom guided exercises of the interactive support and awareness tool of FIG. 1A;

FIG. 13 is one embodiment of a self assessment input user interface and the data output of the interactive support and awareness tool FIG. 1A;

FIG. 14A is one embodiment of a user interface of the triggers submodule located in the observations core module of the interactive support and awareness tool of FIG. 1A;

FIG. 14B is another embodiment of a user interface of the triggers sub module located in the observations core module of the interactive support and awareness tool of FIG. 1A;

FIG. 14C is yet another embodiment of a user interface of the triggers submodule located in the observations core module of the interactive of FIG. 1A;

FIG. 15A is one embodiment of a visualization user interface of the states elements located in the observations core module of the interactive support and awareness tool of FIG. 1A;

FIG. 15B is another embodiment of a visualization user interface of the user state elements located in the observations core module of the interactive support and awareness of FIG. 1A;

FIG. 15C is one embodiment of a states categorization file component user interface located in the observations core module of the states submodule associated with FIG. 15A;

FIG. 15D is one embodiment of a check-in user interface of a states element associated with FIG. 15A;

FIG. 15E is another embodiment of a user input for the check-in element user interface of a states element associated with FIG. 15A;

FIG. 15F is one embodiment of a states element updating user interface associated with FIG. 15A;

FIG. 15G is another embodiment of a states submodule updating user interface utilizing the tagging system associated with FIG. 15A;

FIG. 15H is another embodiment of the states submodule updating user interface associated with FIG. 15A;

FIG. 15J is one embodiment of a searching user interface associated with FIG. 15A;

FIG. 15K is one embodiment of a reminders user interface associated with FIG. 15A;

FIG. 16A is one embodiment of a user interface to visualize core self by user selected time frame and user selected sub state of the interactive support and awareness tool 1A;

FIG. 16B is another embodiment of a user interface to visualize core self of the interactive support and awareness tool of FIG. 1A;

FIG. 16C is yet another embodiment of a user interface to visualize core self of the interactive support and awareness tool of FIG. 1A;

FIG. 16D is yet another embodiment of a user interface to input the user self assessment of core self of the interactive support and awareness tool of FIG. 1A;

FIG. 17A is one embodiment of a user interface to visualize the user insights core module of the interactive support and awareness tool of FIG. 1A;

FIG. 17B is another embodiment of a user interface to visualize the user insights core module of the interactive support FIG. 1A; and

FIG. 17C is yet another embodiment of a user interface to visualize the user insights core module of the interactive support and awareness tool FIG. 1A.

It should be understood that the proportions and dimensions (either relative or absolute) of the various features and elements (and collections and groupings thereof) and the boundaries, separations, and positional relationships presented there between, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments. The following descriptions are not intended to limit the embodiments to one preferred embodiment. To the contrary, it is intended to cover alternatives, modifications, and equivalents as can be included within the spirit and scope of the described embodiments as defined, for example, by the appended claims.

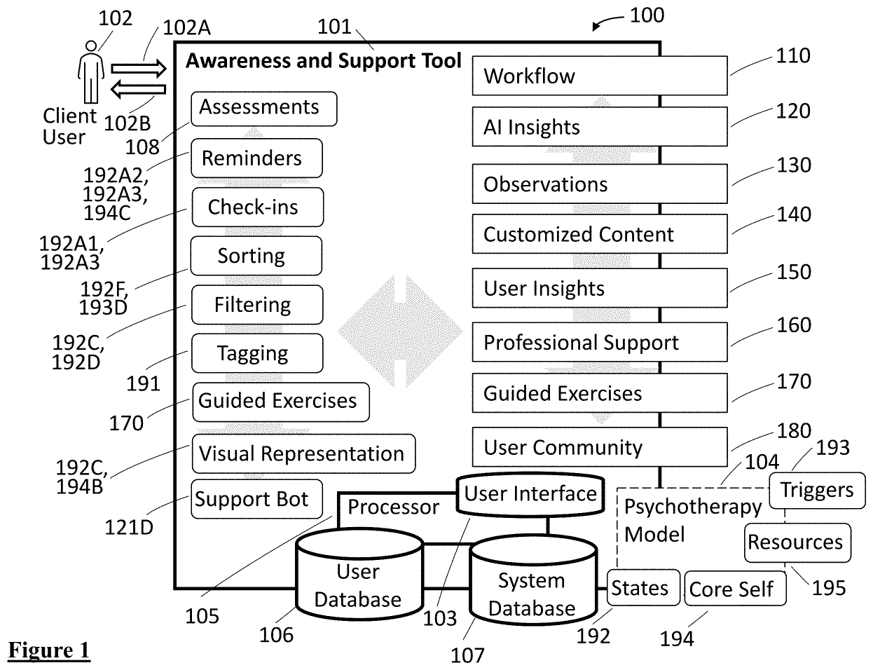

A mental health system with awareness and support tool is disclosed which provides an integrated, personalized, and dynamic system and method to improve a user's mental health. The mental health system (also referred to as “system”) and mental health system method of use (also referred to as “method”) will be discussed with respect to FIGS. 1-17.

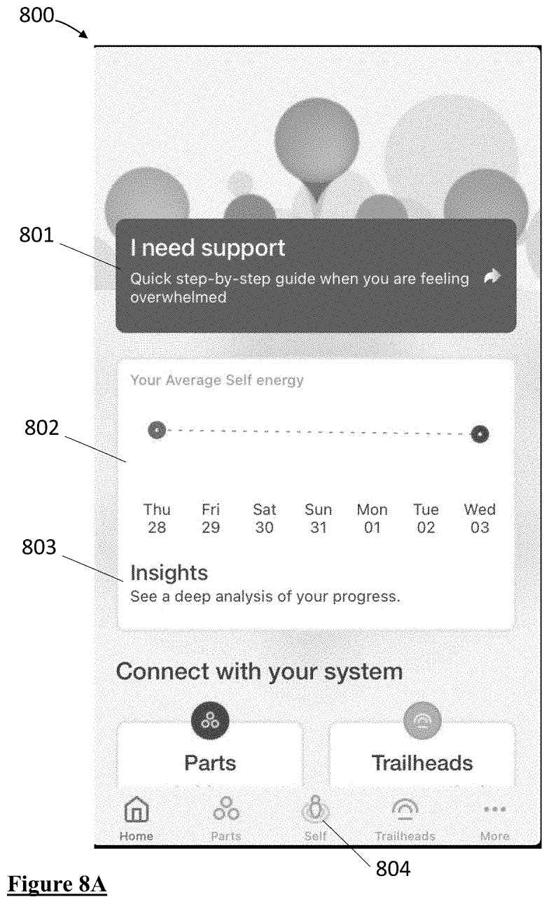

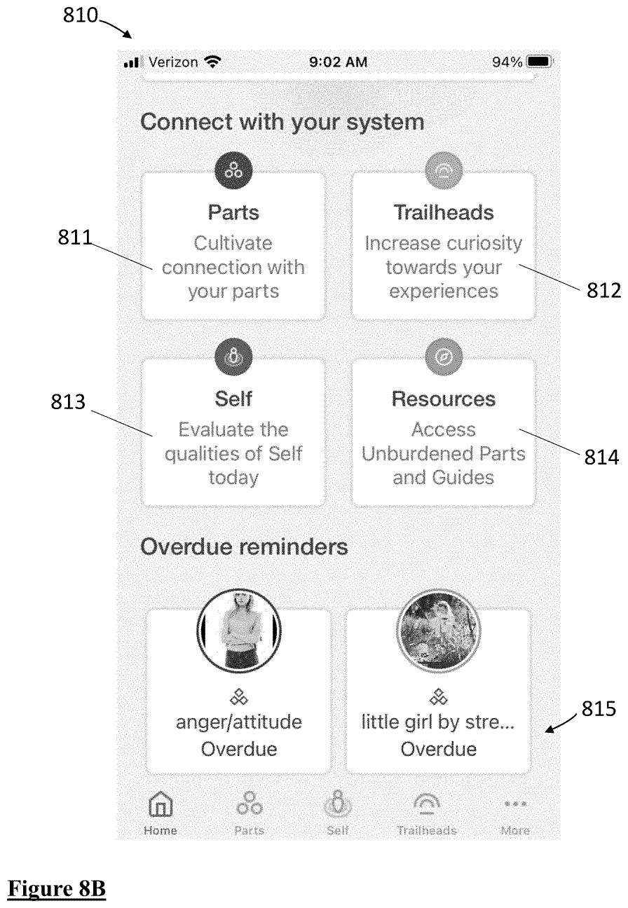

With attention to FIG. 1, a mental health system 100 and is depicted, comprising a mental health awareness and support tool 101 (also referred to as the “awareness and support tool” or simply the “tool”). A client user 102 (also referred to as “user”), such as a user undergoing emotional distress or simply a user seeking to manage their mental health, interacts with the awareness and support tool 101 by way of one or more user interfaces 103 (examples and functionalities discussed, e.g., below with respect to FIGS. 8-17). A processor 105 controls and operates the awareness and support tool 101, to include construction and management of a user database 106, providing and enabling a personalized or customized set of functionalities for a particular user 102, and a system database 107. The system database 107 stores, among other things, educational documentation, a set of standard guided exercises and access to other supportive resources. Aspects of the awareness and support tool 101 leverages one or more psychotherapy models 104 known to those skilled in the art.

Generally, the awareness and support tool 101 comprises a set of eight (8) core modules (collectively, the “tool core modules” or simply “core modules”) depicted in FIG. 1 as a vertical set of eight components or modules on the right hand side of the figure. Specifically, the awareness and support tool 101 comprises core modules of: workflow 110, AI insights 120, observations 130, customized content 140, user insights 150, professional support 160, guided exercises 170, and user community 180. All or a subset of these eight tool core modules may interact with one another, as indicated by the double-headed vertical arrow connecting the tool modules.

A user 102 engages with or operates on one or more core modules with aid of and/or recognition of one or more of nine (9) integration tools depicted in FIG. 1 as a vertical set of nine components or modules on the left hand side of the drawing. Specifically, the awareness and support tool 100 comprises integration tools of: assessments 108, tagging system 191, reminders 192A, 192A3, 194C, check-ins 192A1, 192A3, sorting 192F, 193D, filtering 192C, 192D, visual representation (aka mapping) 192C, 194B, guided exercises (aka meditations) 170, and support bot 121D. All or a subset of these nine integration modules may interact with one another, as indicated by the double-headed vertical arrow connecting the integration modules. One or more of the sets of integration tools may interact with one or more of the sets of eight core modules, as indicated by the double-headed horizontal arrow disposed between these respective sets of modules. For example, workflow 110 may interact with one or more of assessments, reminders, tagging, meditations, and support bot. AI Insights 120 may interact with one or more of assessments, check-ins, tagging, and meditations. Observations/journaling 130 may interact with one or more of reminders, check-ins, filtering, mapping, tagging, and meditations. Customized Content 140 may interact with one or more of assessments, reminders, tagging, meditations, and support bot. User insights 150 may interact with one or more of assessments, reminders, check-ins, filtering, and tagging. And professional support 160 may interact with one or more of assessments and tagging.

One or more of the integration modules, and/or one or more of the core modules, are implemented and/or controlled substantially, completely, and/or at least partially by the processor 105.

The user 102 provides an input 102A of emotional distress at a first level and receives an output 102B, from the awareness and support tool 101, of emotional distress at a second level less than the first level leading to improved mental health. The decreased emotional distress is enabled by one or more benefits of the awareness and support tool 101, e.g., new and enriched awareness, new and enriched support, and decreased barriers to change. A large portion of the disclosure details how a user 102, confronted with emotional distress as input 102A into the awareness and support tool 101, receives outputs 102B from and interacts with the awareness and support tool 101 which result in a decreased and/or mitigated level of emotional distress. The unique functions and features of the awareness and support tool 101, as described, enable this decreased and/or mitigated level of emotional distress.

The user 102 and/or therapist interacts or engages with the awareness and support tool 101 through one or more user interfaces 103 described below. The phrase “user interface” or “UI”, and the phrase “graphical user interface” or “GUI”, means a computer-based display that allows interaction with a user with aid of images or graphics.

The user database 106 and system database 107 may be any database known to those skilled in the art that provides capabilities to include filtering data, sorting data, visually displaying data, capturing data, extrapolating data, etc. The user database 106 and system database 107 (collectively, a “mental health database”) may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The awareness and support tool 101 may be a physical platform comprising computer hardware, computer software, and the like, or may be partially a virtual platform. For example, the awareness and support tool 101 may comprise components that are “in the cloud” meaning generally that some elements may exist physically removed from others and are accessed through a computer network connection, such as by way of a data center. As a specific example, all or part of one or both of user database 107 ad system database 107 may reside in the cloud.

In one embodiment, the awareness and support tool 101 is a portable electronic device, such as a laptop, smart phone, or a desktop computer. In one embodiment, the user accesses and/or interacts with the awareness and support tool 101 remotely, such as through cloud-computing.

The mental health systems 100 through the awareness and support tool 101 receives a dysregulated and/or stressed system of an individual or community and creates a self-regulated and harmonized system of an individual 181, 182. As depicted in FIG. 1A, a client user 102 provides or inputs (self) assessments 108 and observations 130 into the awareness and support tool 101 (utilizing integration tools such as tagging 191 and the core workflow module 110), which are used or processed by the processor 105 to create or build or develop AI insights 120, user insights 150, select custom content 140 or custom guided exercises 170 based on the user input. The insights 120 are shared with both the client user 102 and, if desired the professional 160.

The AI insights 120 may utilize cross-referencing, tagging (via the tagging system 191), relational context, sentiment analysis and other meta-data provided to the user database 106. The AI insights 120 may comprise, relative to the data input by the client user 102, aggregations, correlations, AI predictions, classifications, and visualizations. The insights, as generated by the processor 105, are unique to the client user 102 and dynamic in that the insights change over time given additional user input, and further analysis (by the processor 105).

The phrases “intelligent system,” “artificial intelligence,” “bot” or “Bot,” and “AI” mean a machine, system or technique that mimics human intelligence. The phrase “machine learning” means a subset of AI that uses statistical techniques to enable machines to improve at tasks with experience. The phrases “neural networks” and “neural nets” means an AI construction modeled after the way adaptable networks of neurons in the human brain are understood to work, rather than through rigid predetermined instructions.

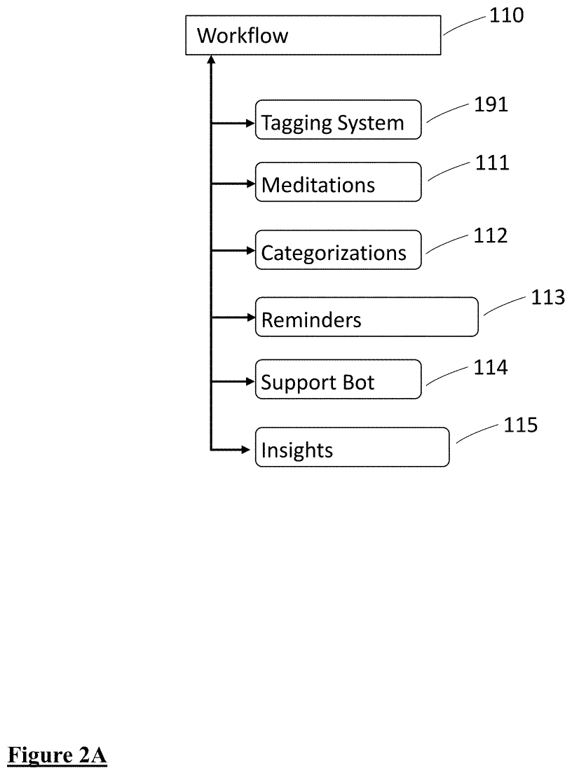

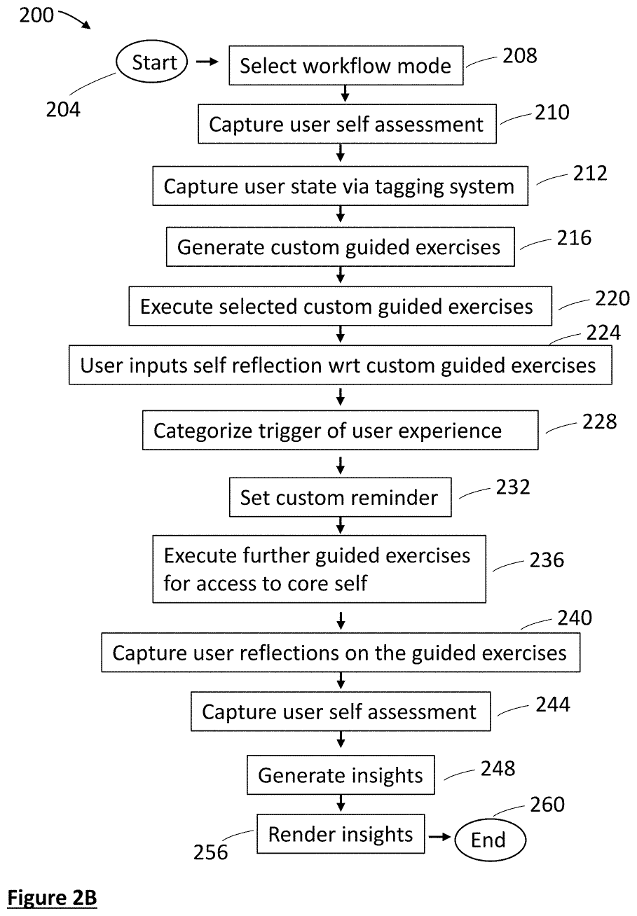

The awareness and support tool 101 will now be described in detail relative to the set of core modules and the set of integration tools, the features and functions of the awareness and support tool 101 enabled by or created by the processor 105 and the user database 106 maintained by the processor 105. FIGS. 2-7 provide further details and description of the core modules and integration modules. FIGS. 8-13 provide further details and description of the user interfaces of the awareness and support tool 101, as created, rendered, and controlled by the processor 105.

FIG. 2A depicts the workflow 110 core module of the awareness and support tool 101. Workflow 110 generally receives input from a user 102 as to observations regarding a distressing experience and provides interactive, customized output to the user which guides the user 102 through the distressing situation while capturing and clarifying the user observations in a supportive and structured fashion. These client user 102 data inputs are captured by the processor 105, by way of one or more user interfaces 103 described below, and stored in the user database 106. The processor 105 creates or generates the customized output from the user (observation) input and stores these outputs to the user database 106. Existing approaches to capturing client experiences, if performed at all, are by way of therapist-client conversations and/or pen and paper j ournaling in a non-structured manner (meaning, among other things, a lack of customized support at the time of capturing experiences such as just a blank piece of paper).

Workflow 110 interacts with or uses the tagging system 191 integration module to input, broadly, the state of the user 102, such as, e.g., emotions, beliefs, body sensations, visuals, dreams, states, qualities of core self, and behavioral urges. The term “state” or “user state” means characteristics of the user, such as, for example, emotions, beliefs, body sensations, visuals, dreams, states, behavioral urges, etc.; more generally, a user state includes user mental state and user physical state

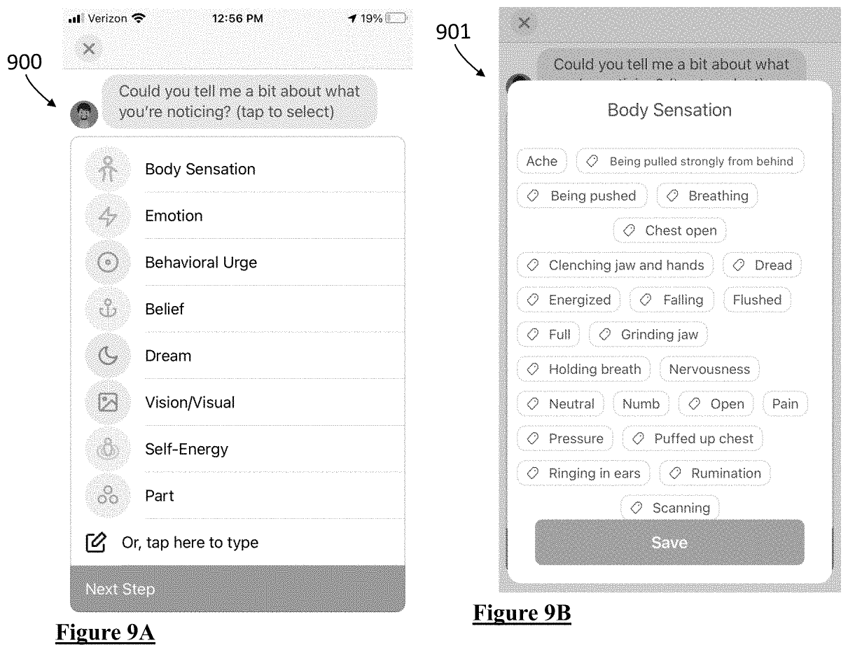

The tagging system 191 allows the user 102 to readily and easily input the entirety of their distressing experience because it allows for quick clicks of commonly used inputs. These user inputs aka tags to the tagging system 191 are received by the processor 105 and stored in the user database 106. The processor 191 may draw from these user inputs to, e.g., allow the awareness and support tool 101 to categorize, filter, and search for specific items of the user experience both within workflow 110 or within any other module or component of the awareness and support tool 101 such as the observation core module 130 in either list or visual mode. Additionally, the tagging system 191 will allow for the AI insights 120 (or User insights 150) to be generated utilizing processes such as sentiment analysis among other things which fuels the generation of custom content 140 and custom guided exercises 170. Among other things, the tagging system 191 decreases time needed to capture a user experience (using tags) versus writing everything out (via pen and paper, physical flags such as post-it notes, etc.) which may lead to decreased barriers to change like anxiety, confusion and being overwhelmed. (See FIGS. 9A-B for one embodiment of a user interface of the tagging system 191).

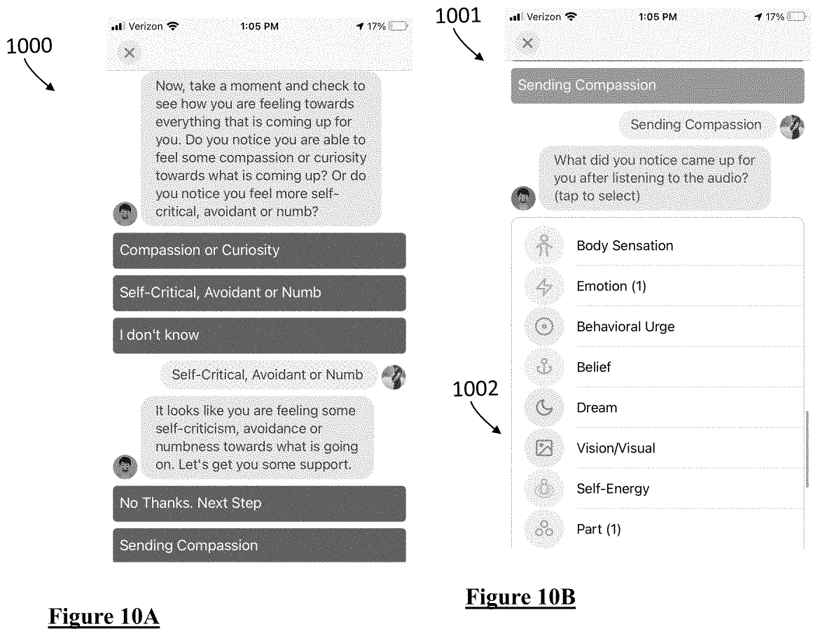

Guided exercises 170 are verbal and/or audio vignettes used to address a particular user state or (distressing) experience. A user 102 selects guided exercises 170 which appear to be beneficial to or resonate with their particular state. The set of guided exercises are stored in the user database 106 and/or the system database 107 and accessed and managed by the processor 105. Additionally, the user 102 can be offered a list of custom guided exercises in the workflow 110 or the custom content 140 of the awareness and support tool which are generated by user inputs such as assessments 108 and tagging 191 analyzed by the processor 105 and stored in the user database 106. The recommended guided exercises as delivered by the custom content core module 140 and are designed to fit into where the particular user is in the stage of emotional regulation and also leverages the AI insights core module 120. In existing approaches, users are left to an unintegrated experience of self-regulation and have guided exercises (and associated journaling) in a separate place. The user must decipher which guided exercise may be right based off what they are experiencing in the moment and where they are in their (mental health) work. Note that the set of guided exercises 170 comprise both a full standard set of guided exercises stored in the system database 107 and a set of customized guided exercises compiled from the full set of guided exercises stored in the system database 107 (See FIG. 10B for one embodiment of a user interface of guided exercises 111).

Categorization 112 receives input from a user 102 so as to categorize a particular user experience under a previous experience or label the experience as a new experience. The categorization is then associated with the set of (tagged) inputs described above and stored by the processor 106 in the user database 106. (See FIG. 11A for one embodiment of a user interface of Categorizations 112).

Reminders 113 receive input from the user as to if/when a user would like to be reminded to return to a trigger and have a particular user provided label. For example, a user may have a particular trigger that is associated with partner relations, the trigger thereby labeled as, e.g., “relationship with partner.” The reminder 113 may comprise a date and time to come back to a particular triggering experience. The tool 101 may be configured to automatically remind the user 102 to come back and revisit the particular trigger.

Support Bot 114 may provide user customized encouraging messages and reminders regarding work or efforts that the user is undertaking. The support bot is managed by the processor 105 and may comprise AI algorithms or techniques.

Insights 115 provide one or more new relationships or observations seen in the AI insights core module 120 or the user insights core module 150, as developed by the processor 105, as to the data provided by the user. For example, the user may input a rating of self-perception of access to core self at the beginning of a particular session (with a therapist and/or with the awareness and support tool 101) and at the end of the particular session, such that a percentage of increase or decrease in the amount of perceived access to core self may be presented. The difference in such a percentage would provide a measure of the results of using the Workflow 110 core module. Other insights of Insight 115 are possible, such as being able to suggest which of the nine integration tools the user has utilized in the past that have shown to be helpful to the user (See FIG. 13 for one embodiment of a user interface of Insights 115).

The processor 105 of the awareness and support tool 101 handles, integrates, manipulates, maps, and renders data stored in the user database 106 and the system database 107. All or some of the data stored in the user database 106 and the system database 107 may be entered by a user 102 by way of the user interface 103.

Data entered by the user 102 may be temporally-marked, meaning the data entered is marked with time and date of entry in the case of discrete data inputs (e.g., a user input as to a particular user state mental attribute will be marked with the date and time of entry) or duration of activity (e.g., a user begin and end time and date when executing a guided exercise). Data entered by the user 102 may include: i) user state data, such as any attribute of the user's mental state (e.g., emotions and beliefs such as happy, sad, angry), physical state (e.g., heart racing, sweating, jaw clenched), behavioral urges (e.g., wanting to yell), visions (seeing a small child hiding), and dreams; ii) use trigger or trailhead data, such as category or name of trigger or trailhead and attributes associated with the trigger or trailhead; iii) user self-assessment data, such as a sliding scale data entry as described below with respect to FIG. 13; and user self-reflection data (see e.g. step 224 of FIG. 2B). The user may additionally, or alternatively other data as described in the disclosure. Other means of user 102 data entry are possible additionally or alternatively to the user interface 103, such as by may of Wi-Fi or other wireless connection to remote devices. In one embodiment, some or all of the user provided data is not temporally-marked. Data entered by the user may also be termed “current” vs. “existing” meaning, respectively, data entered during a present session or engagement with the awareness and support tool 101, vs. data already entered (and residing on one or more of the user database and system database) during a previous (and now not active or closed) session or engagement with the awareness and support tool 101.

The user database 106, in connection with the processor 105, may operate on the temporally-marked user data to store the data, sort the data, and/or filter the data based on selected criteria. For example, the user, through the user interface 103 or otherwise, may seek to sort to find all triggers that are associated with a mental state of anger, or to filter to locate all periods of a physical state of clenched jaw, etc. The user database 106, builds up a collection of user data provided over time, thereby storing both previously-entered or pre-existing user data and data entered by the user during a current engagement with the awareness and support tool 101.The system database 106, in connection with the processor 105, may operate on meditations aka guided exercises.

FIG. 2B presents one embodiment of a method 200 of use of the workflow 110 core module of the awareness and support tool 101 of FIG. 2A. However, aspects and features of any of the FIGS. 1-18 may be referenced to enhance the disclosure of the method of use. The method 200 will be described with a sample use case of a user undergoing an emotionally distressing experience. The use case is in no way limiting to the method.

Generally, the method 200 starts at step 204 and ends at step 260. Any of the steps, functions, and operations discussed herein can be performed continuously and automatically. In some embodiments, one or more of the steps of the method of use 400, to include steps of the method 400, may comprise computer control, use of computer processors, and/or some level of automation. Indeed, many of the steps of method 200 are performed automatically, principally if not entirely by processor 105. A user interacts with or performs one or more of the described steps using a display/GUI. The steps are notionally followed in increasing numerical sequence, although, in some embodiments, some steps may be omitted, some steps added, and the steps may follow other than increasing numerical order.

The following is a case example presenting one embodiment of a method 200 of use of the workflow 110 core module of the awareness and support tool 101 of FIG. 2A. Kira is a 25 year old bank teller living in Birmingham, Ala. She is currently in a serious committed relationship and is struggling. She can't seem to connect to her partner emotionally even though she very much wants to. Kira is self-motivated, likes challenges to her creativity, and utilizes technology multiple times a day to make life easier for her. She is open to therapy to help with her relationship issues but is concerned about the cost of therapy, not having time for it in her schedule, not interested in the “old school” way of doing anything and doesn't want anything to do with people being fake. She has grown up in an era where she has access to every answer under the sun with the internet and needs help “helping herself” with where she is stuck.

Kira is experiencing a crunch on her time due to her therapist's limited availability, a strain on her finances because therapy is not cheap and is starting to grow frustrated with not being able to clearly understand how to help herself in-between sessions. All sorts of psychotherapy tools are randomly pieced together and given to her. Some are pen and paper, some are random guided exercises, some are books, and others are just stand alone websites. There is nothing that she is able to access under the conventional way of working that can support Kira in a comprehensive way in between sessions with her therapist.

Kira is having a particularly hard day on the day she downloads the awareness and support tool 100. In the conventional, current approach, Kira would just get out her old pen and paper journal when she got home and try and just get some feelings out. This would lead to frustration because she doesn't feel supported as she is navigating what is coming up for her. She closes her journal after just a few moments and goes back to her old ways of coping such as TV, food or substances. She has to wait until she is able to see her therapist again in a few weeks to feel like she can really make some progress creating increased distress and frustration that she can't help herself in the way she would like. Self-transformation is hard enough as it is, and she is frustrated that she can't get assistance in-between sessions in a way that feels best for her.

After starting at step 204, the method 200 proceeds to step 208. At step 208, a user selects Workflow mode, meaning the user 102 engages the Workflow 110 core module of the awareness and support tool 101. In one embodiment, as depicted in FIG. 8A, the user 102 selects (e.g., through a touch-screen of the UI of the display of the tool 100, as generated and rendered by the processor 105) the “I need support” portion 801 of a home screen of the tool 100. In Kira example, Kira views the home screen and the portion 801 as an immediately calming feature. After completing step 208, the method 200 proceeds to step 210.

At step 210, the user 102 inputs self-assessment of their state with respect to the particular emotionally distressing experience (termed the “particular experience” or simple the “experience”). Such a self-assessment may be through a sliding scale measure from low to high, or from 0 to 100, as depicted in UI 1300 of FIG. 13. After completing step 210, the method 200 proceeds to step 212.

At step 212, the user 102 inputs further user state data via the tagging system 191. These data are captured by the processor 105 and stored in the user database 106. The data input may include various grouping or sets of categories, such as body sensation, emotion, behavioral urge, belief, dream, vision/visual, qualities core self (Self), states (Parts), or user customized groupings. Such groupings may be presented or rendered (by processor 105) as depicted in UI 900 of FIG. 9A. One or more of the data groupings associated with the use state inputs may have pre-populated descriptors or tags that may be tagged by the user, as depicted for the body sensation grouping depicted in UI 901of FIG. 9B. The pre-populated descriptors may include a fixed set of groupings (e.g., Full, grinding jaw, etc.) and may include one or more descriptors set or defined by a particular user. The user selects or tags one or more of the groupings (as depicted in FIG. 9A) and associated descriptors of that grouping (as depicted, for the Body Sensation grouping, in FIG. 9B). (The user may select, in this example and/or in any one or more scenarios, embodiments, systems, or methods of the disclosure, by way of a touch screen input, or any other means of UI input known to those skilled in the art, received by the processor). The groupings described may be associated with one or more psychotherapy models 104, and may, among other things, enable filtering (e.g., 192C, 192D), mapping, and/or sorting (e.g., 192F, 193D) by the processor 105 of the user inputted data.

Kira, by easily being able to capture her experiences as to how she is feeling or what she is noticing about her (emotionally distressed) state or experience, feels a sense of new and enriched support. Note that the data input at step 212 is used by the processor to construct a unique and temporally marked file set, or a unique and temporally marked collection or set of data as to the particular distressing experience of the particular user, such a file set enabling the tool 101, by way of the processor 105, to build or add to a set of mappings and associations of the data or the data set which provide unique insights, functions for the user. Such associations and/or insights are described below and throughout the disclosure with respect to FIGS. 1-17.

After completing step 212, the method 200 proceeds to step 216.

At step 216, custom guided exercises are generated or accessed (by processor 106) based on the data input by the user 102 in step 212 and/or further user input as to user state. For example, as depicted in UI 1000 of FIG. 10A, the user 102 additionally select or enter their choice of pre-populated options (those of FIG. 10A include “Compassion or Curiosity” and “Self-Critical, Avoidant or Numb,” and “Sending Compassion”). In the example of FIG. 10A, the user 102 has selected “Self-Critical, Avoidant or Numb.”

Kira realizes that the custom guided exercises are based on her answers to how she is feeling towards her emotional distress, representing a novel approach to support. (Traditionally, clients are journaling with one app that is not niche specific, then after they are finished, they open up another app with generalized guided exercises. The approach of the system 100 represents a significant step forward in the self-transformation journey as it is integrated into a single experience). After completing step 216, the method 200 proceeds to step 220.

At step 220, the user executes the one or more custom guided exercises. After completing step 220, the method 200 proceeds to step 224.

At step 224, the user 102 provides self-reflection input as to the one or more custom guided exercises as executed in step 220. As depicted in FIG. 10B, a custom guided exercise associated with “Sending Compassion” has been executed (the guided exercise having an audio component) and the user 102 is presented with groupings for data entry. The groupings 1002 presented are similar to those presented in FIG. 9A.

Kira is supported with self-reflection (at this step 224) after she has completed her guided exercise, as captured in the custom workflow experience, she is creating within the tool. Stated another way, the tool 101, by way of the processor 105, is constructing a workflow for a particular user (e.g., Kira) based on the user inputs. After completing step 224, the method 200 proceeds to step 228.

At step 228, the user enters a categorization of the particular distressing experience (for Kira, the experience is associated with her relationship with her partner). The categorization is one of two options: a new trigger (trailhead) or an existing trigger (trailhead). FIG. 11A depicts one UI 1101 to receive user selection of this categorization. FIG. 11B presents a UI 1102 wherein a user 102 opts to enter a new trigger (trailhead) for the particular experience, here shown as a “Test Trailhead.”

Kira would have a trigger that would be entitled “relationship with partner”. She is offered the option to add this reflection to either a current trailhead or a new one. This also represents a novel approach to be able to seamlessly and effortlessly capture and categorize separate experiences under certain triggers (trailheads). The conventional way of doing this ranges and with pen and paper individuals are usually left using tabs or color coding entries often representing a very frustrating and overwhelming way of self-transformation. After completing step 228, the method 200 proceeds to step 232.

At step 232, the user may set a custom reminder as to the trigger just considered. This functionality enables a user to be encouraged to work between therapy sessions and enables a more effective user management of mental state by, e.g., facilitating the active management of mental state. Kira views her work with the tool 101 as presenting a new and enriched awareness that provides a new and enriched way of continuing the work in-between sessions by being offered a custom reminder for her to keep check-in back in with her distressing experience. (Conventionally, there is no way to seamlessly and easily continue the diligent and necessary work of continuing to return to an experience which is distressing for users but essential to self-transformation.) After completing step 232, the method 200 proceeds to step 236.

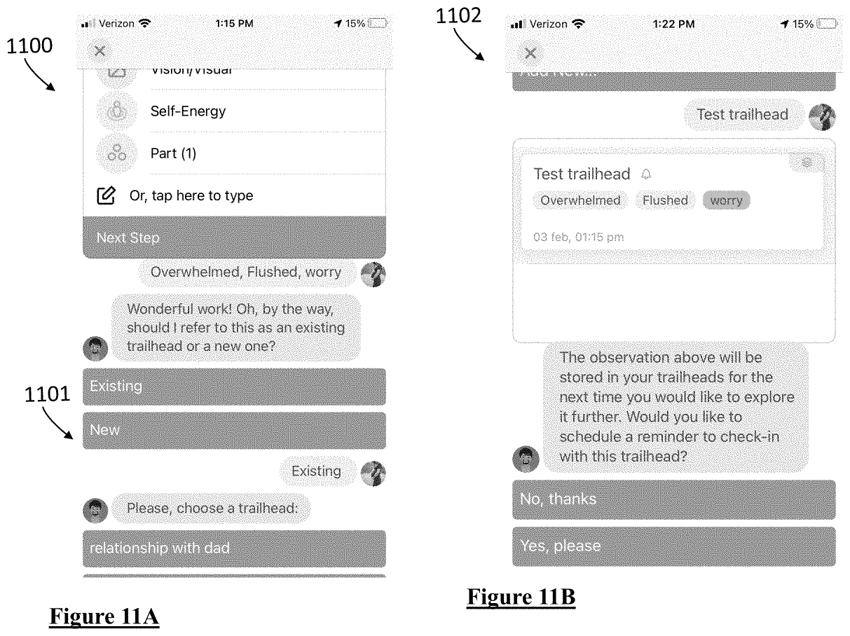

At step 236, the user 102 is offered and executes any additional custom guided exercises selected by the user (see UI 1203 of FIG. 12B). These guided exercises or guided experiences are uniquely selected by the AI insights core module 120 and/or the User insights 150 of the support and awareness tool based off user inputs during previous user sessions. After completing step 236, the method 200 proceeds to step 240.

At step 240, the user 102 may enter user reflections on the guided exercises executed in step 236. Kira is encouraged with the new and enriched support in being able to capture her reflections on the guided exercises right in the workflow 110 which may be used later for insights into what was helpful for her during her distress. After completing step 240, the method 200 proceeds to step 244.

At step 244, the user 102 inputs self-assessment of their state with respect to the particular emotionally distressing experience as they have now spent time working with their distressing experience. similar to step 210. See FIG. 13 and UI 1301. After completing step 244, the method 200 proceeds to step 248.

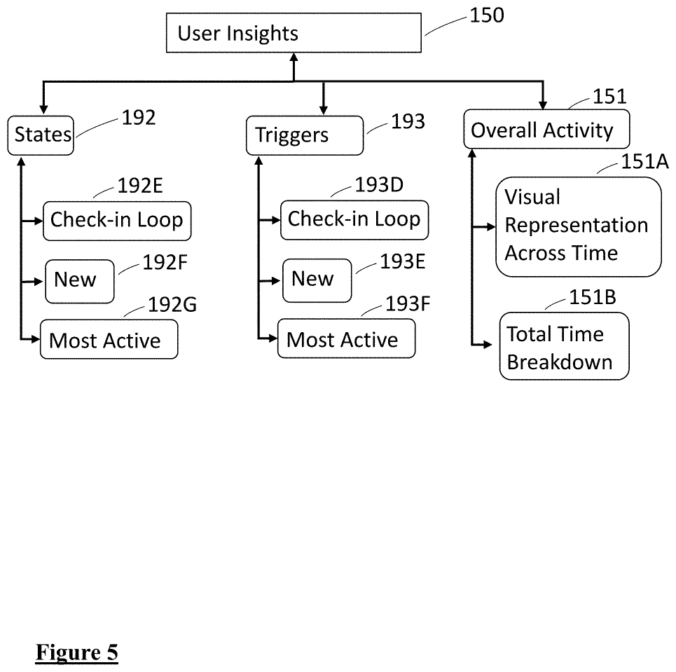

At step 248, the processor 105 of the tool 101 generates insights. For example, the processor 105 may compare the self-assessment data of step 210 with that entered in step 244 to determine an increase in emotional well-being (as caused by the user 102 executing the method 200) to generate an insight as to the percentage increase in emotional well-being (see UI 1302 of FIG. 12). Insights 120 are described in more detail below, e.g., with respect to FIG. 5.

Once Kira inputs her self-assessment, she is given new and enriched awareness into just how helpful it is when she spends time working on her self-transformation in-between sessions. (This may be analogized to someone on a weight loss journey. The person steps on the scale before the workout session and after and when they can see a tangible loss, it is motivating and encouraging to keep the journey going. Often emotional distress and consequently the journey to emotional well-being can be very intangible; the tool 101 offers many tangible ways to see and engage with the process, decreasing many barriers to change including frustration, exhaustion and confusion). After completing step 248, the method 200 proceeds to step 256.

At step 256, the processor 105 of the tool 101 renders insights. Insights 120 are described in detail below, e.g., with respect to FIG. 5. After completing step 256, the method 200 ends at step 260.

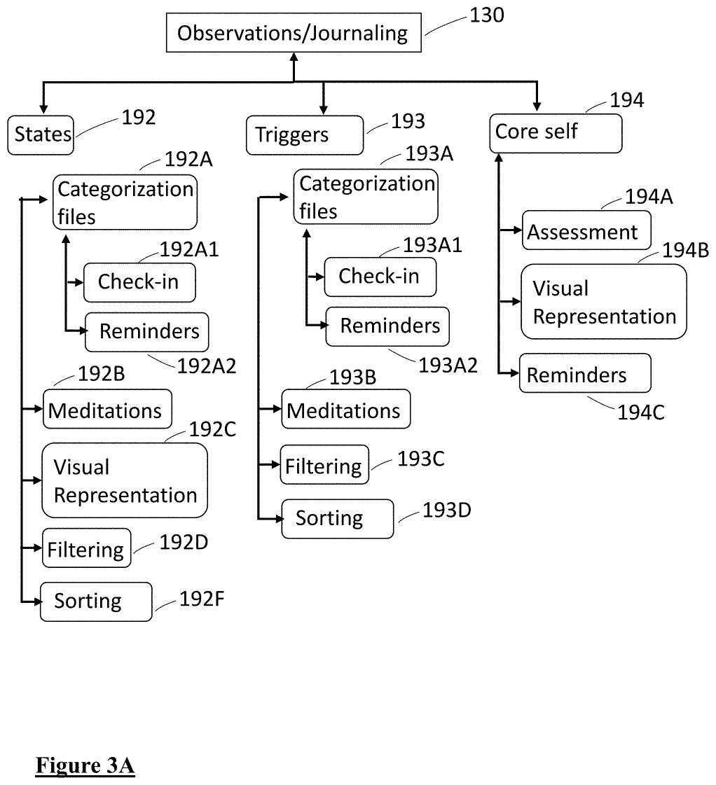

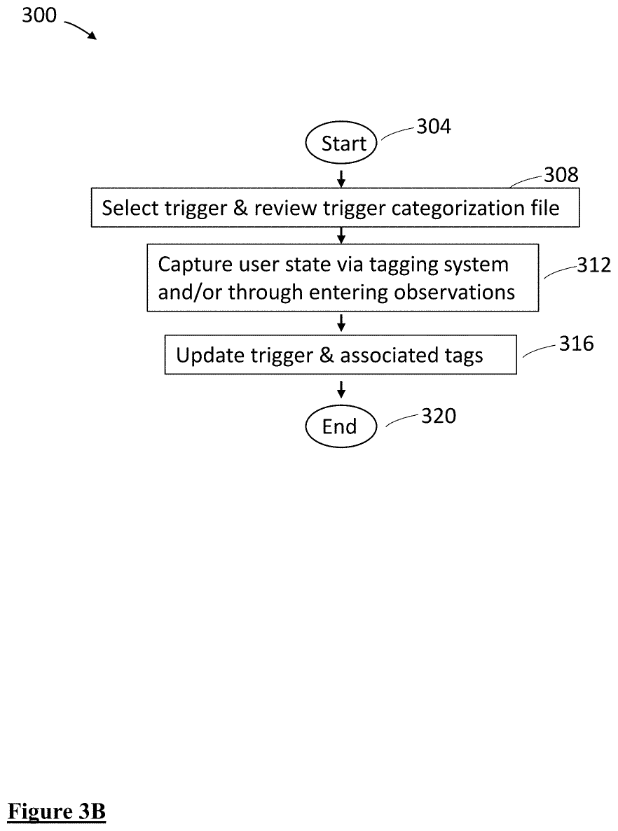

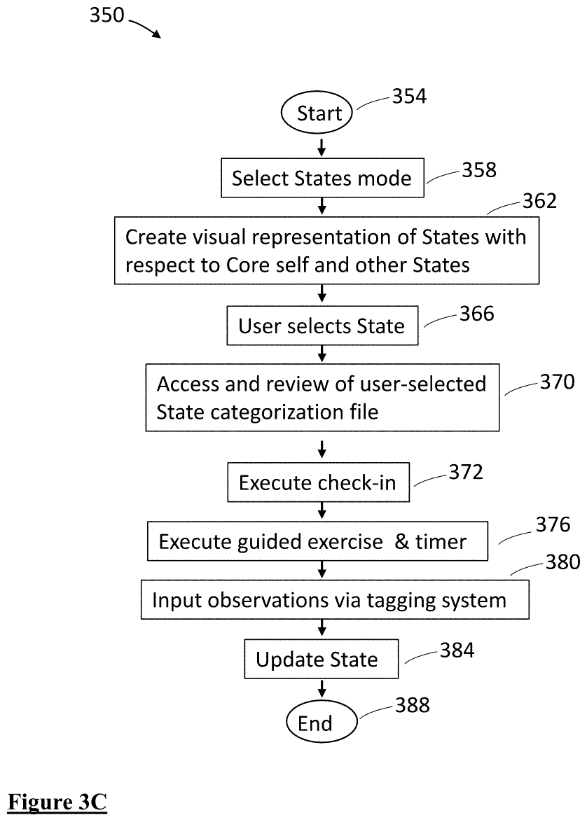

FIG. 3A depicts the observations/journaling 130 core module of the awareness and support tool 101. Observations/journaling 130 generally utilizes one or more of the nine integration tools such as assessments 108, reminders, tagging 191, support bot 121D, and others and captures information about states of distress 192, core self 194, resources 195 and triggers 193.

The operations of the observations/journaling 130 core modules, as with all core modules and all integration tools, is performed by the processor 105, typically as enabled by data stored in one or both of the system database 107 and user database 106, with all or some aspects presented to the user on the user interface 103.

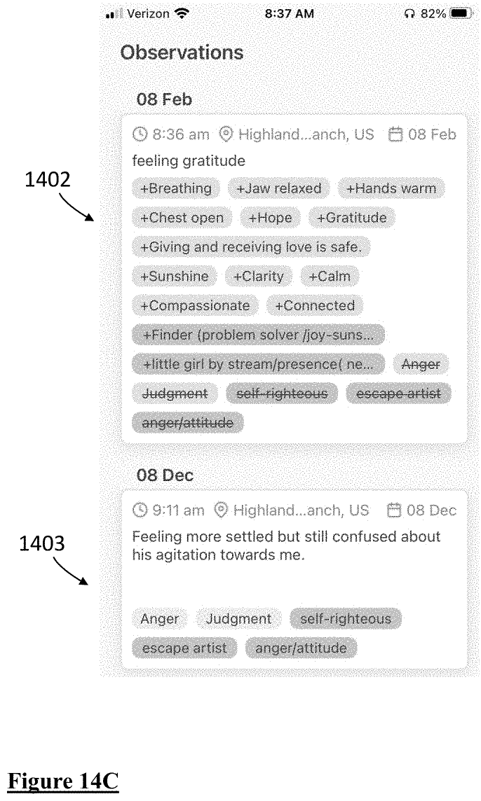

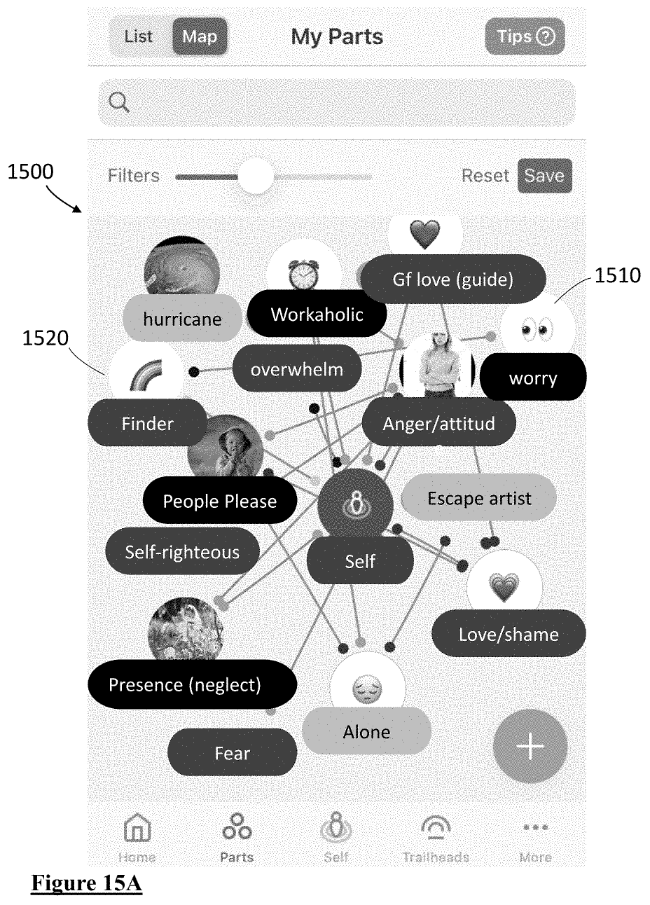

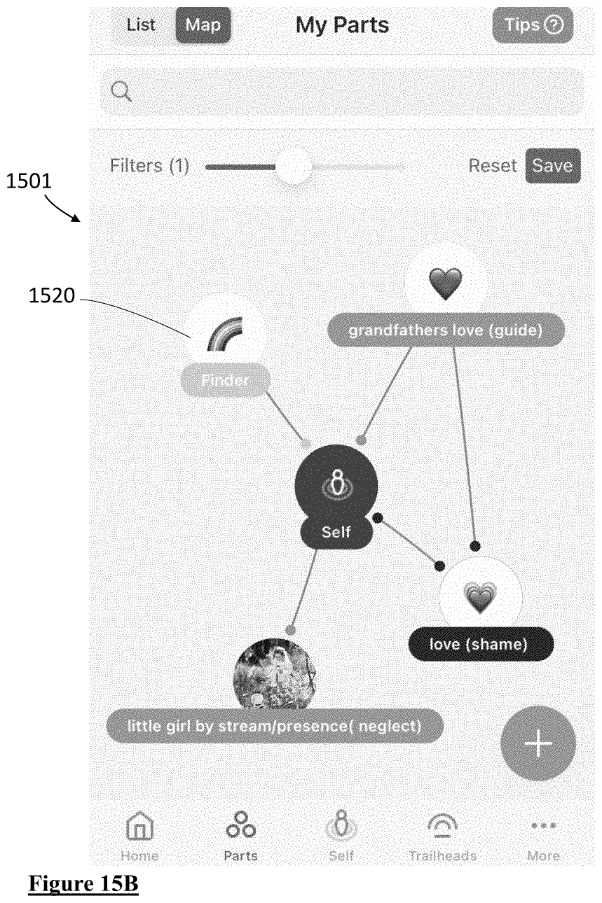

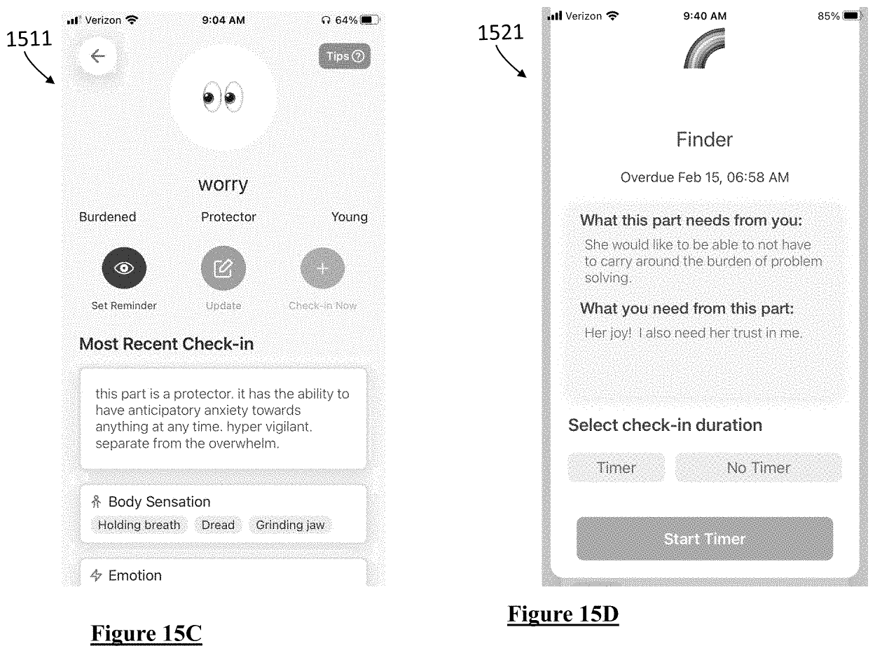

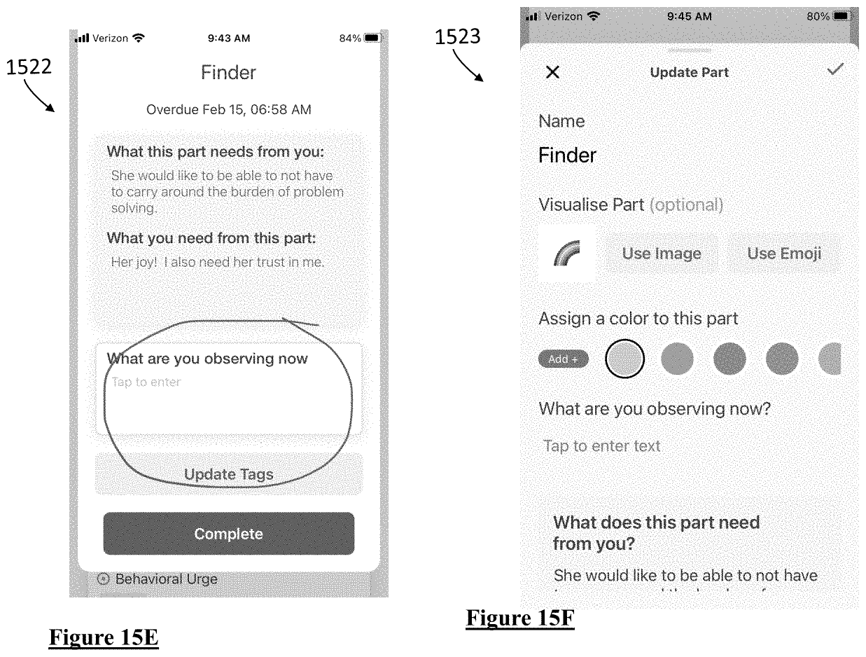

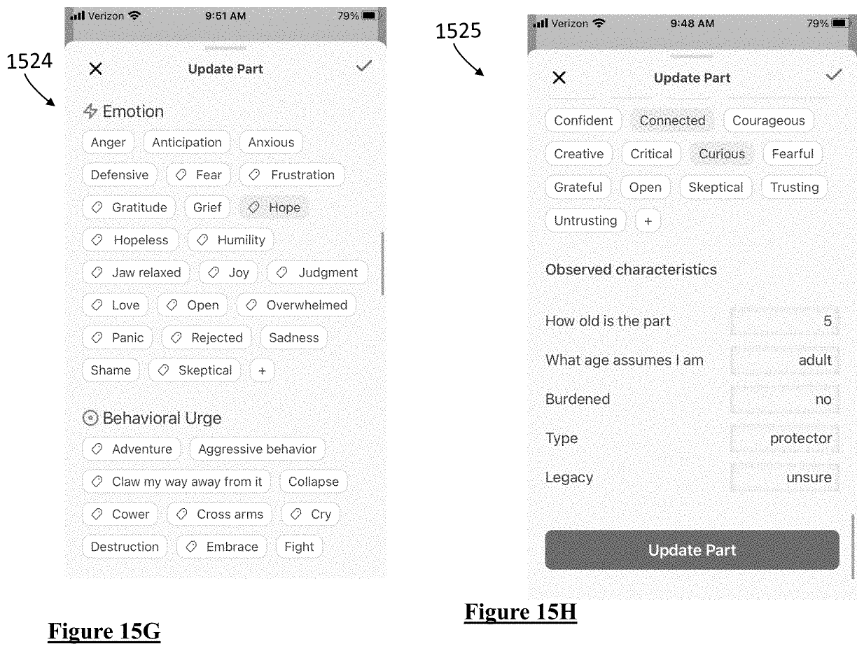

States of distress 192 are associated with information and observations of a particular user 102. For example, a particular client user 102 may possess the emotion of sadness, a worry part, a finder part, a self-righteous part, sweaty palms, anger, rumination, a desire to run away etc. (See FIG. 15A, described below). The observations core module 130 provides a depository to clearly categorize and interact with states of distress through custom categorization, reminders (e.g., 192A2, 192A3, 194C), supported check-ins (e.g., 192A1, 192A3), and guided exercises 170, and enable; a user to filter states of distress by tagged data or attributes (e.g., by a user observation of a feeling or user state such as anger). A result or output of the observations core module 130 is increased clarity, confidence, and curiosity for a user 102 when working with their states of distress. Many current approaches to managing or working with states of distress are performed through pen and paper techniques or through attempts to adapt systems, such as mind mapping/brain storming system, which are not focused on self-transformation work and therefore are very limited in effectiveness.

The observations core module 130 allows a user to discover what states they interact with most, see a list of all states, and filter by assigned tags 192D or other identified characteristics, or provide custom visualization for their states 192 C. This way, a user may explore specific aspects of their inner experience or the entire experience at once. This represents a new and enriched tool for working with the user 102 and their identified states and triggers.

The states are categorized into user specified categories 192A. In these user defined categories data may comprise data both generic or common to a particular state and stored in the system database 107, and data specific to a particular user and stored in the user database 106. In one embodiment, the information pertaining to the user states of distress and subsequent user defined organization and categories for those states are solely stored solely in the user database. In existing systems or methods, such states data is “stored” by pen and paper. FIG. 15C depicts an example states (Parts) categorization file for the “worry” part. The processor 105 receives, manages, and/or manipulates the states categorization file data.

The states check-in 192A1 receives from the user 102 observations in the present moment regarding the state and integrates pertinent information regarding the state and a place to easily note updated observations. A user 102 may enter or check-in with the state by entering, e.g., observations regarding that state (See FIG. 15E and element 1522). The check-in 192A1 enables structured support for checking in with a given user state.

The state reminders 192A2 provide a date and time for a reminder to the user to check-in with a state and to be taken directly to the user defined category page. The user 102 is provided a reminder per their set specifications to check-in with the state. Existing systems or methods include user setting reminders, if at all, on a stand-alone portable electronic device (e.g., a smartphone) or journaling by pen and paper. There is no supportive tagging and no history that documents trajectory of a state or trigger nor is there any system incorporating it into a larger support and awareness tool.

The state guided exercises 192B provides to the user 102 a selectable list of guided exercises to help facilitate the check-in with the part, thereby providing supportive guidance while checking in with the state of distress. The states guided exercises 192B provides, among other things, increased calm, confidence and clarity checking in with a state in between sessions with a professional.

The state visual representation 192C allows the user 102 to manipulate all aspects of their identified states to personalize it to their liking, to include, e.g., relationships between states, relationships between states and the core self, and visual placement of states in a relative and absolute manner. See FIGS. 15A-B. Such a visual representation of a user's internal system comprised of states and core self, as driven by or enabled by underlying user data where the user can easily extrapolate, manipulate and excavate the data to advance their self-transformation is not available in existing systems.

The state filtering 192D allows a user 102 to filter states by specific tags (e.g., words or phrases) to provide a new and enriched experience through interacting with their inner experience. Such filtering may reveal new relationships, new drivers, and/or new connections between states, triggers, etc. that are otherwise unobservable. For example, as depicted in FIG. 15J, a user may type in the word fear into states filtering 192D to determine triggers associated with fear (in the example of FIG. 15J, the trigger “alone” is found to be associated with fear.)

The state sorting 192E allows a user 102 to sort their list of states by user selected parameters such as most recent or alphabetical. This provides a new and enriched experience for a user 102 to access their inner experience as such information reveals new information regarding the recent interactions with states that are otherwise unobservable in visualized form. For example, a user 102 may feel that they are in a state of anger most of the time, however, with the awareness and support tool 101 they see from the observations module 130 and the submodule of the states 192 utilizing the sorting tool 192F they see they actually most recently accessed a state of avoidance, not anger. This provides an objective visualization of the interactions in their inner experience vs a subjective nature which is traditionally how this is exercised.

The trigger submodule 193 comprises and interacts with categorization files 193A, guided exercises 193B, filtering 193D, and sorting 193E modules or components or elements. The categorization file 193A submodule of the triggers module 193 comprises a check-in 193A1 submodule and a reminders 193A2 submodule.

The triggers integration module 193 receives user inputs such as upsetting emotions, beliefs, body sensations, visualizations, states, qualities of core self, and behavioral urges, and sets reminders, performs check-ins, filters, sorts and searches for certain similar qualities across multiple triggers, and offers custom guided exercises to help support areas of healing specified by user 102 data input stored in the user database 106. The triggers module193 serves to capture and clarify the journey of self-transformation from distress to emotional wellness of a user's most triggering experiences, resulting in a user feeling less overwhelmed, more confident, clear and calm at continuing work in between therapy sessions. In contrast, conventional approaches require users to journal on their own with no structure or support, resulting in users feeling overwhelmed with where to start capturing what is going on, commonly resulting in a user doing nothing which can create more avoidance and prolongs healing.

The trigger categorization file 193A receives user input such as upsetting emotions, beliefs, body sensations, visualizations, states, qualities of core self, and behavioral urges and enables a user to be guided through capturing important aspects about their triggering experience. The trigger categorization file 193A also provides a central location to document all experiences and the trajectory associated with triggering situations. Further, users may quickly access supportive sub features such as reminders 193A2 and check-ins 193A1 from this output. Additionally, the trigger categorization file 193A allows users to see the trajectory of their healing with a history report of all observations, with items crossed out that are no longer applicable, symbols to identify newly added tags. In contrast, conventional approaches require users to journal on their own with no structure or support (such as reminders), without supported check-ins that are integrated into the entire experience and without the advanced visual representation depicting the trajectory for a triggering experience that the awareness and support tool 101 provides.

The trigger check-in 193A1 receives updated observations about a triggering experience and incorporates custom guided exercises selected by the user. A user 102 may enter or check-in with the triggering experience by entering, e.g., observations regarding that trigger. The trigger check-in 193A1 enables structured support for checking in with a given trigger and easily categorizes experiences under a particular trigger via the tagging system.

The trigger reminders 193A2 operate per user specifications (e.g., provide a date and time) for a reminder to the user to check-in with a triggering experience. Existing systems or methods include user setting reminders, if at all, on a stand-alone portable electronic device (e.g., a smartphone) or journaling by pen and paper. There is no supportive tagging and no history that documents trajectory of a trigger.

The trigger guided exercises 193B is a list of guided exercises selected by the AI Insights core module 120 and/or user insights 150 module based off information in the user database 106. For example, Kira might be given a guided exercise of how to work with her anger as she has selected anger as a frequent state she experiences in relationship to her trigger of her relationship with her partner. The processor 105 receives a user 102 selection of a particular customized guided exercise specifically aimed at helping the user at this section of the self-transformation journey. Existing systems which involve guided exercises are not integrated into the user experience and set specifically for where they are at in their journey, typically contained in disparate places from multiple sources.

The trigger filtering 193C receives user selected tags of which filtering is sought, e.g., filtering by fear, tightness in chest, etc. The ability to filter triggers by states, e.g., enables and provides increased clarity and confidence when a user is experiencing a feeling of being overwhelmed because it is very valuable to discover or see patterns between triggers and states. Such discovered patterns, such as the number of different triggers associated with different emotions, reveals ways that a user typically displays distress that are otherwise unknown or not naturally available. Another example filtering result is to identify all relevant triggers that have particular tags, i.e., certain body states, or behavioral urges.

The core self module 194 comprises and interacts with assessment 194A, visual representation 194B, and reminders 194C submodules or components or elements.

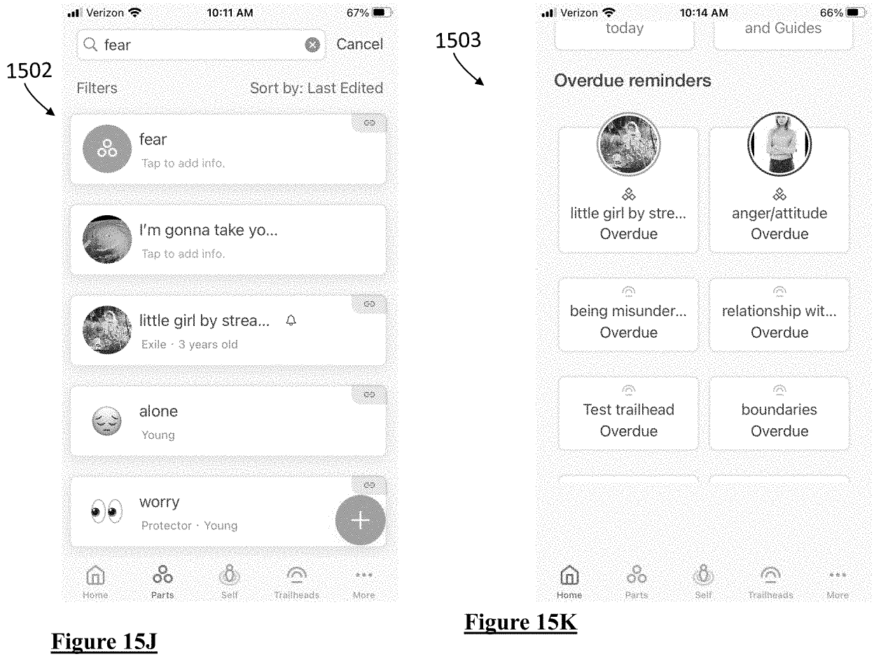

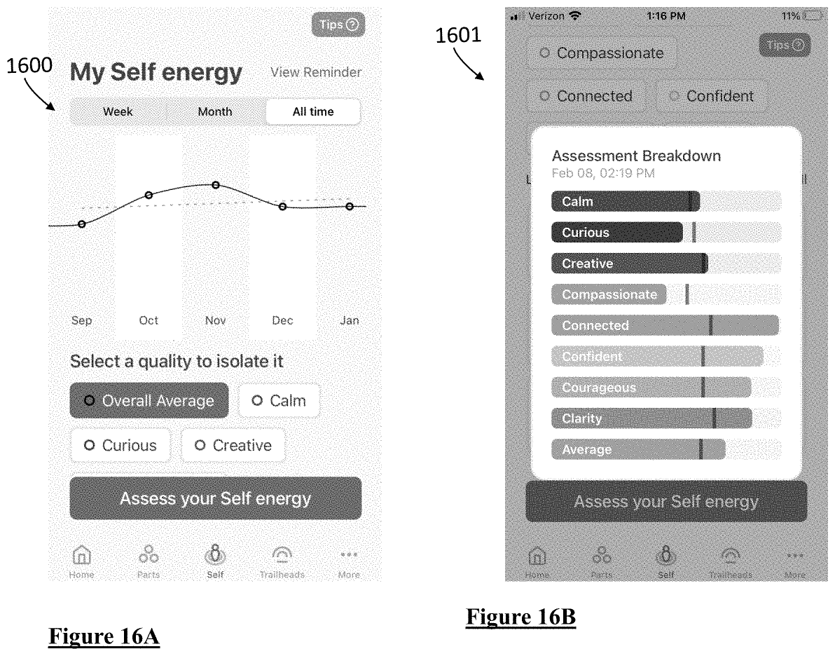

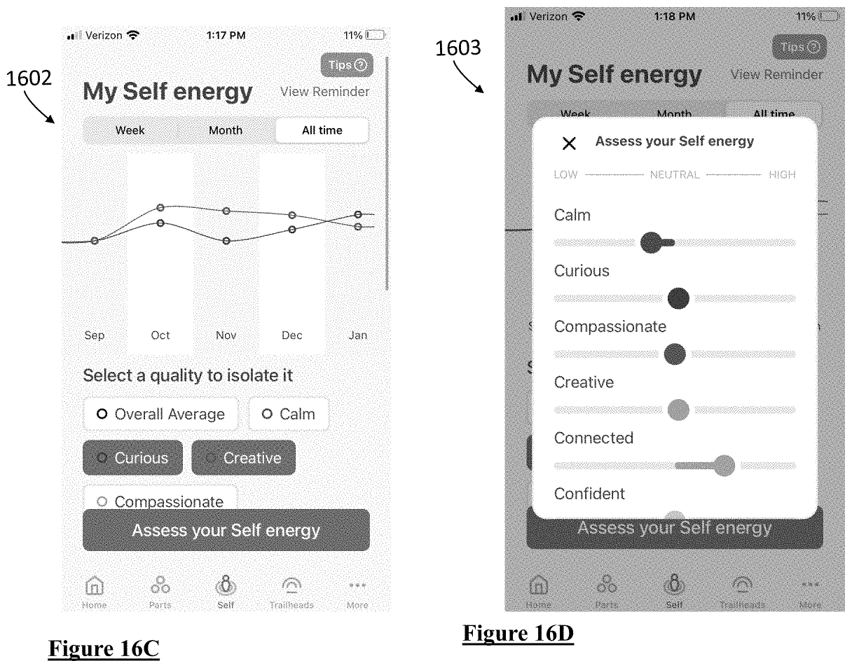

The core self submodule 194 receives from a user the user's perceived level of access to core self (defined above) so as to visually represent an internal experience and provide clarity and encouragement to a user 102. See FIGS. 16A-D. Existing systems or methods at best may record a snap-shot measure of core self but do not record changes over with time or have the ability to select certain aspects of core self to be seen in comparison to other aspects of core self or the experience of core self overall.

In one embodiment, core self (Self) comprises the eight components identified in FIG. 16A: calm, curious, creative, compassionate, connected, confident, courageous, and clarity.

The core self assessment 194A module receives user-felt levels of access to each core self quality with time.

The core self visual representation 194B receives a user's inputted data about perceived levels of each core self quality, represented over specified periods of time (week, month, year, etc.) to produce or render a visual representation of the trajectory of the user's access to each of these qualities internally over time. See, e.g., FIG. 16C.

The core self reminder 194C receives date and time and how often the user would like to be reminded to check-in with their perceived access to each of the core self qualities, thereby making a user's tracking of mental well-being progress much more effective and easier, and providing an increased awareness of progress.

FIG. 3B presents one embodiment of a method 300 of use of the trigger 193 integration module of the observations 130 core modules of FIG. 3A. However, aspects and features of any of the FIGS. 1-18 may be referenced to enhance the disclosure of the method of use. The method 300 will be described with the same sample use case of user Kira as employed with respect to FIG. 2B. The use case is in no way limiting to the method.

Generally, the method 300 starts at step 304 and ends at step 320. Any of the steps, functions, and operations discussed herein can be performed continuously and automatically. In some embodiments, one or more of the steps of the method of use 300, to include steps of the method 300, may comprise computer control, use of computer processors, and/or some level of automation. Indeed, many of the steps of method 300 are performed automatically, principally if not entirely by processor 105. A interacts with or performs one or more of the described steps using a display/GUI. The steps are notionally followed in increasing numerical sequence, although, in some embodiments, some steps may be omitted, some steps added, and the steps may follow other than increasing numerical order.

Continuing on her self-transformation journey, Kira leaves a recent session with her therapist and takes a moment to use the awareness and support tool 101 to capture information and observations about a recent trigger (trailhead) she worked with during the session.

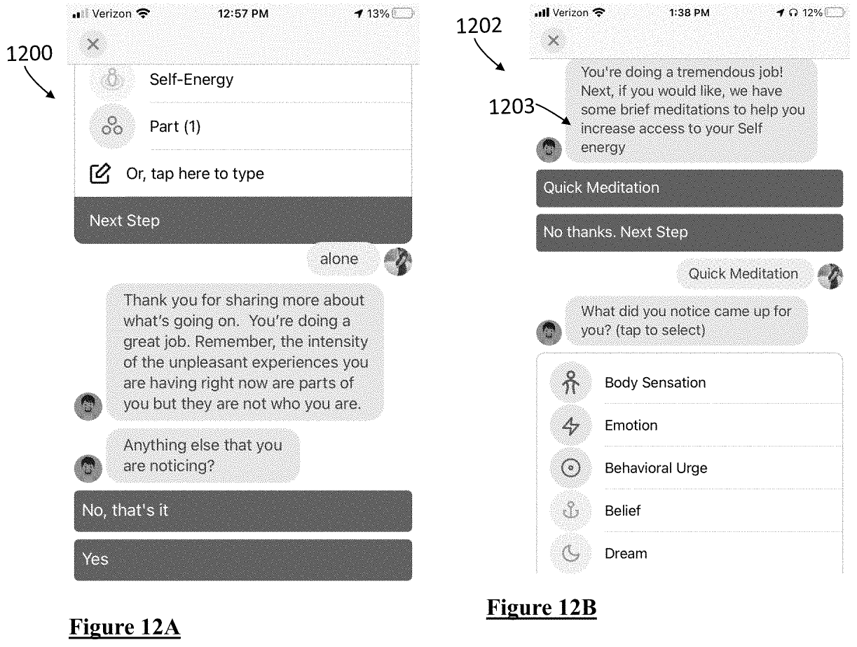

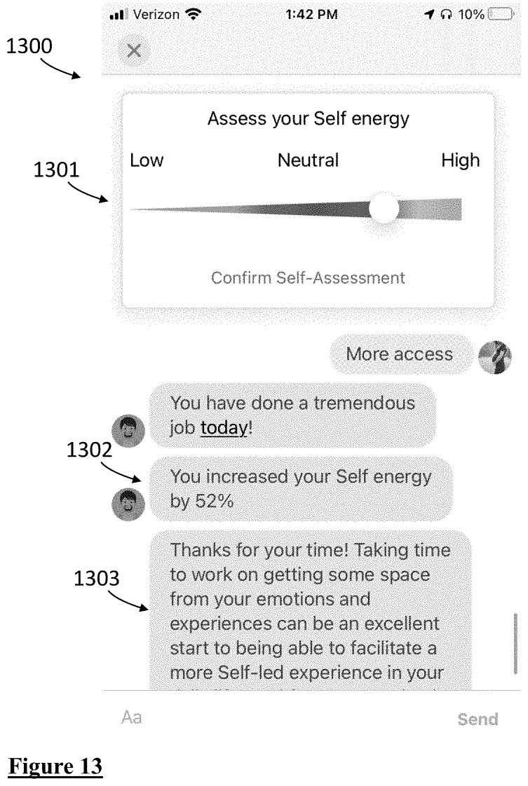

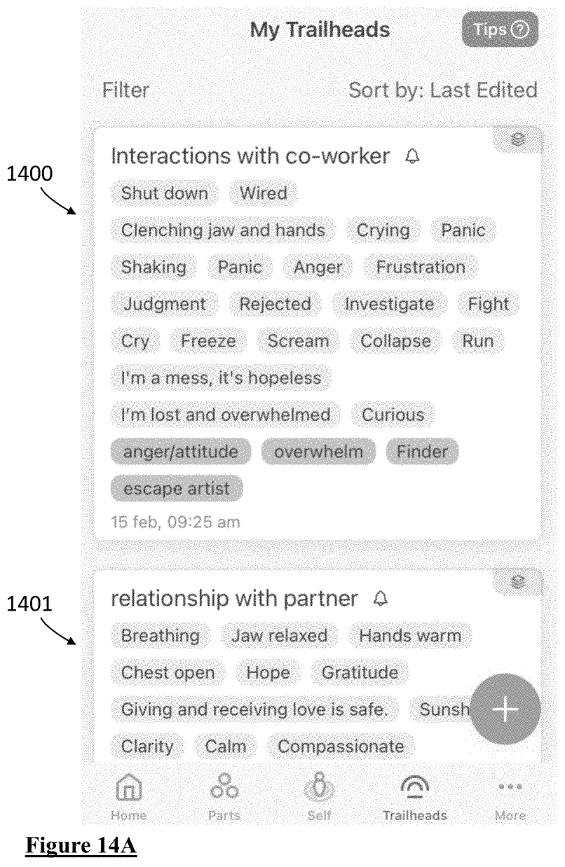

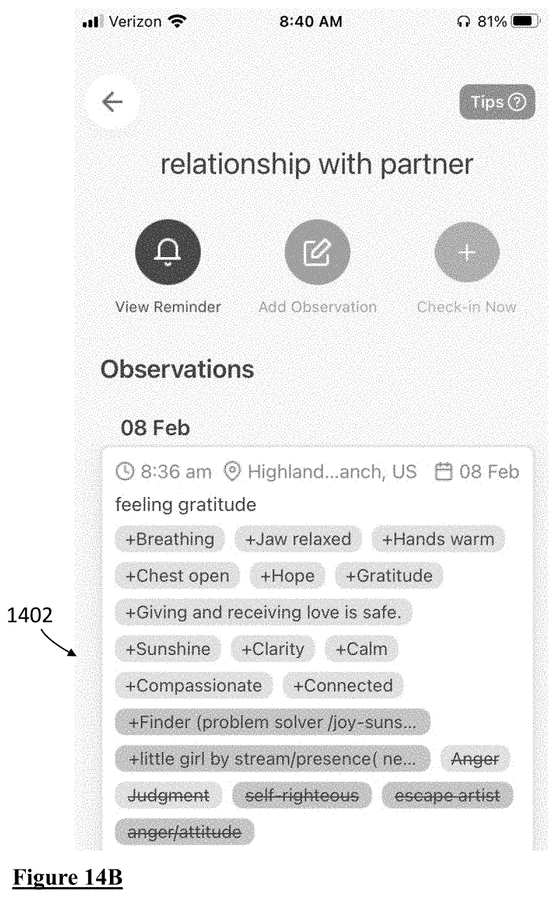

After starting at step 304, the method 300 proceeds to step 308. At step 308, a trigger (trailhead) is selected (See FIG. 8B and element 812, wherein element 812 is activated or engage by the user) and the associated trigger categorization file (trailhead bio) is reviewed. As depicted in FIG. 14A, two triggers (trailheads) are presented: Interactions with Co-worker (see FIG. 14A and element 1400), and (partially shown) Relationship with Partner (see FIG. 14A and element 1401)

Kira is able to quickly update an existing trigger (trailhead) (i.e., the Relationship with partner trailhead) in the awareness and support tool 101 by navigating to the triggers (trailheads) button on the home screen (FIG. 8B, element 812). Kira is quite pleased with this (trigger/trailhead) feature as traditionally she would have to wait until she got home to capture this information and instead, she is seamless in entering data in the parking lot before returning to work. Being able to capture these experiences in the awareness and support tool allows her system to find a sense of peace and calm knowing she does not need to take up any mental space trying to remember everything that happened during her recent therapy session. From the home screen Kira is taken to step 308 which takes her to a list of her recent triggers (trailheads) (see FIG. 14 and elements 1400 and 1401), and she opens the categorization file (trailhead bio) for the (Relationship with partner) trigger (trailhead). After completing step 308, the method 300 proceeds to step 312.

At step 312, the state of the user 102 is captured. The user 201 selects from the collection or set of traits or elements for the selected trigger (trailhead), e.g., one or more of the traits or elements associated with the Relationship with partner trigger (trailhead), such as breathing, jaw relaxed, etc. (See FIG. 14A and element 1401). The user may also enter text observations with respect to the selected trigger (trailhead) (see FIG. 14C and element 1403).

Kira is able to easily interact with her selected trigger that she experiences over and over through utilizing the tagging system 191, setting reminders 193A2, providing updates or checking in 193A1 to see how she is feeling regarding this trigger (trailhead). Through visual representation she is able to see the progress of her work with this particular trigger (trailhead) utilizing the tagging system 191. She can see certain items in bold that are new to that recent entry and previously tagged items are crossed out (see FIG. 14B and element 1402). This creates a new and enriched way of working with a trigger (trailhead) as traditionally individuals only know they are working with a trigger when they are flooded with emotions and are not able to see the trajectory of healing in such a complete way. With the awareness and support tool 101, she is able to easily see changes in her emotions, body sensations, beliefs, states (parts), etc. (e.g., through the strike outs versus bold elements within the observation portion of her selected trailhead—see FIG. 14B and element 1402). This offers her hope and optimism because when one is constantly dealing with triggers it can feel like nothing ever changes as the traditional approach presents such an intangible experience. After completing step 312, the method 300 proceeds to step 316.

At step 316, the particular trigger (trailhead) and associated tags are updated. After completing step 316, the method 300 ends at step 320.

FIG. 3C presents one embodiment of a method 350 of use of the states (parts) 192 module of the observations 130 core module of FIG. 3A. However, aspects and features of any of the FIGS. 1-18 may be referenced to enhance the disclosure of the method of use. The method 350 will be described with the same sample use case of user Kira as employed with respect to FIGS. 2B and 3B. The use case is in no way limiting to the method.

Generally, the method 350 starts at step 354 and ends at step 388. Any of the steps, functions, and operations discussed herein can be performed continuously and automatically. In some embodiments, one or more of the steps of the method of use 350, to include steps of the method 350, may comprise computer control, use of computer processors, and/or some level of automation. Indeed, many of the steps of method 350 are performed automatically, principally if not entirely by processor 105. A interacts with or performs one or more of the described steps using a display/GUI. The steps are notionally followed in increasing numerical sequence, although, in some embodiments, some steps may be omitted, some steps added, and the steps may follow other than increasing numerical order.

After starting at step 354, the method 350 proceeds to step 358. At step 358, states (parts) mode is selected (see FIG. 8B and element 811, the element 811 selected by the user by any of several means to include a touch-screen UI). After completing step 358, the method 350 proceeds to step 362.

At step 362, the processor 105 creates or generates a visual representation of all states of the user 102 with respect to one or both of core self and other states. See FIG. 15A. The visual representation depicts the relationships between states and between those states and core self.

Kira views her states (parts) map; the visual representation is wonderfully helpful to her as she can see the relationships and layout between her states (parts) and core self (Self) and how they work together, or not, to help increase her well-being. This represents a new and enriched experience of visually representing States (parts). Current representations are made in pen and paper formats and cannot be readily accessed. The pen and paper experiences are cumbersome, frustrating and don't allow for the user to engage in them readily due to these barriers. The visual mapping feature of the awareness and support tool 101 represents removing a barrier to change in this way as the visual representation of the inner system is easily accessed. Additionally, Kira is able to filter her experience and see only a portion of her map. This is a brand new experience as this is not something that can currently be done. She can filter by a trigger (trailhead), e.g., so all states (parts) associated with her frustration with her co-worker show up. See FIG. 15B. She can filter by age, e.g., so all her very young states (parts) can show up. She can see only healed states (parts), etc. These data may also be presented as a list view. (For clarity, the Kira use case is continued at step 366 through the remainder of method 350 after the remainder of the method 350 is generically described). After completing step 362, the method 350 proceeds to step 366.

At step 366, the user 102 selects a particular state. The state may be selected directly by the user 102 from a listing of states, as accessed through the states element 811 of FIG. 8B or may be accessed after user filtering (as described above) to locate a particular state, such as a state associated with a user defined experience. After completing step 366, the method 350 proceeds to step 370.

At step 370, the user accesses and reviews the categorization file associated with the selected state. The categorization file is as described above with regards to FIG. 3A. After completing step 370, the method 350 proceeds to step 372. At step 372, the user 102 executes a check-in, as described above with respect to FIG. 3A. After completing step 372, the method 350 proceeds to step 376. At step 376, the user 102 executes a selected guided exercise with associated timer. After completing step 376, the method 350 proceeds to step 380 wherein the user 102 inputs observations via the tagging system 191. After completing step 380, the method 350 proceeds to step 384 wherein the state is updated. After completing step 384, the method 350 proceeds to step 388 and the method 350 ends.

Returning to the Kira use case at step 366, Kira accesses her Worry Part (element 1510 of FIG. 15A), the worry Part associated with clenching of her jaw. After step 366, Kira proceeds to step 370 wherein she opens up the states categorization file (part bio) page (see FIG. 15C element 1511), then proceeds to step 372 and starts a check-in (See FIG. 15D). After completing step 372, Kira executes step 376 comprising a guided exercise with associated timer (See FIG. 15D). Setting the timer and closing her eyes she is able to access her relationship with this part. She notes what she observes utilizing the easy tagging system at step 380. The states categorization file (parts bio page) allows her to see what more information this part might want to share with her with prompts as to what information states (parts) typically carry (see FIG. 15G). She is also easily able to categorize this part, note its age, and the relationship to Self (See FIG. 15H). As this part of her feels like it is being heard and understood because she feels comfortable checking in with it on her own. She appreciates the custom guided exercises that are offered to her in her check-in, as it softens and allows her Self to take the lead again being about a feeling of mental well-being. She can easily set a reminder to check-in with this part which is an essential part of cultivating relationships between the core self (Self) and states (parts). When these relationships are cultivated there is a greater likelihood that mental well-being can be established and sustained. This represents a new and enriched experience as currently individuals are left to set reminders, if they even do so, to check-in with their states (parts) and they are then forced to head to separate places to capture the observations of the relationship when they are noted. The awareness and support tool 101 allows individuals the ability to capture, enhance, categorize, organize and extract essential information about the nature of these inner relationships which is not seen or able to be done this way. The state (part) that Kira worked with is then updated at step 384 and the method 350 ends at step 388.

Note that a sub workflow may occur as part of the step 370 access and review of user-selected states, wherein reminders are selected, parameters for a new or existing reminder are established, and one or more reminders are generated.

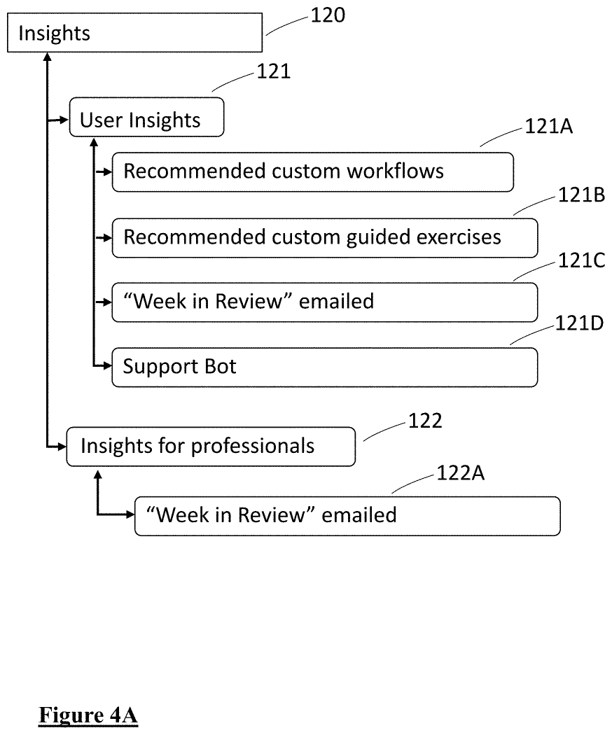

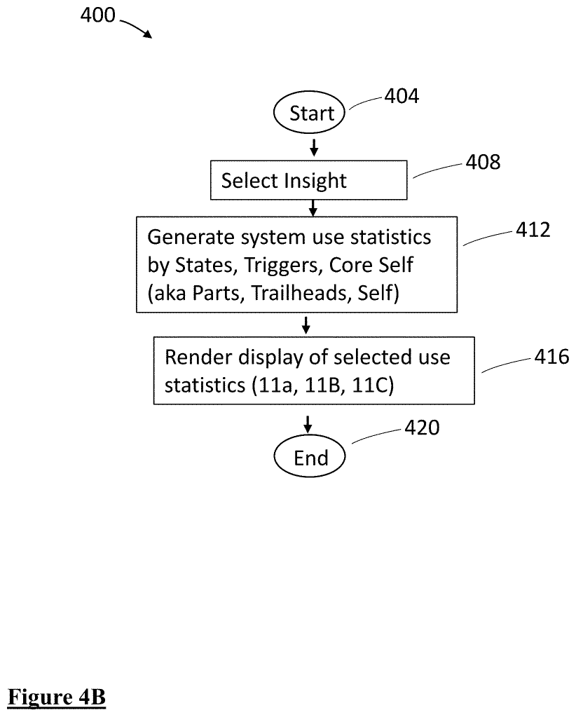

FIG. 4A depicts the AI Insights 120 core module of the awareness and support tool 101. AI Insights 120 comprises insights for the user 121, comprising recommended custom workflows 121A, recommended custom guided exercises 121B, “week in review” 121C, and support Bot 121D, and insights for professional (e.g., a therapist) 122, comprising “week in review” 122A updates.

FIG. 4B presents one embodiment of a method 400 of use of the user Insights 150 module of FIG. 4A. However, aspects and features of any of the FIGS. 1-18 may be referenced to enhance the disclosure of the method of use. The method 400 will be described with the same sample use case of user Kira as employed with respect to FIGS. 2B, 3B, and 3C. The use case is in no way limiting to the method.

Generally, the method 400 starts at step 304 and ends at step 320. Any of the steps, functions, and operations discussed herein can be performed continuously and automatically. In some embodiments, one or more of the steps of the method of use 300, to include steps of the method 300, may comprise computer control, use of computer processors, and/or some level of automation. Indeed, many of the steps of method 300 are performed automatically, principally if not entirely by processor 105. A interacts with or performs one or more of the described steps using a display/GUI. The steps are notionally followed in increasing numerical sequence, although, in some embodiments, some steps may be omitted, some steps added, and the steps may follow other than increasing numerical order.

After starting at step 404, the method 400 proceeds to step 408 wherein the user selects a particular insight, such as average activity times with the awareness and support tool by date. After completing step 408, the method 400 proceeds to step 412.

At step 412, the processor 105 generates system use statistics associated with the user selected particular insight of step 408. In the example of the average activity times insight, the calculations of average activity times per date are calculated. After completing step 412, the method 400 proceeds to step 416.

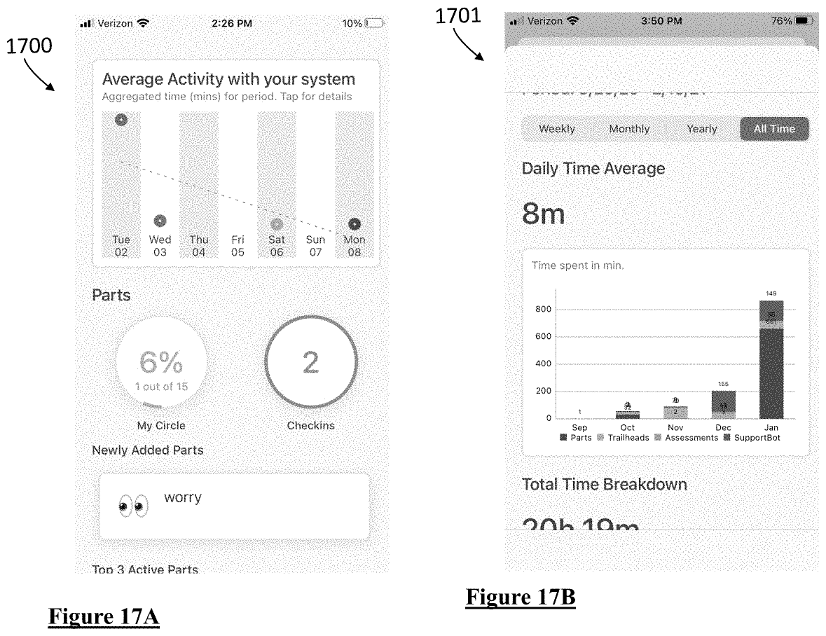

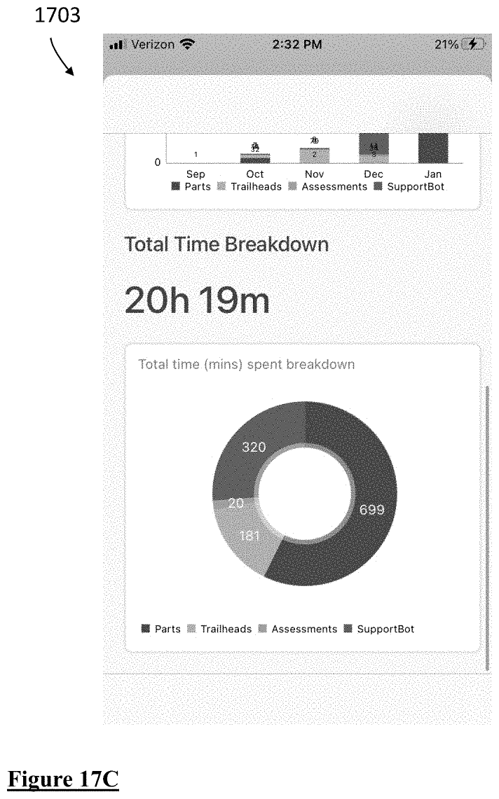

At step 416, the user selected insight is rendered by the processor. In the example of the average activity times insight, a graph of average activity per day is rendered (see FIG. 17A element 1700).

Returning to the Kira use case, Kira utilizes the user insights core module to create new and enriched experiences such as increased awareness and encouragement in her journey of self-transformation. From the home screen (FIG. 8A element 803) she is taken to the insights page where is able to see a detailed overview of her experience of core self (Self), her work with her states (parts) and triggers (trailheads) and her engagement in the system overall (see FIG. 17A). Clicking on the average activity within her system she is able to see a breakdown of the total time she is spent in each area of the awareness and support tool 101 on a weekly, monthly, yearly and all time view (see FIG. 17B). She is also able to view where she has spent her time while she was utilizing the tool 101 which provides her a new and enriched support and awareness to where she is best able to utilize her energy and resources for self-transformation (see FIG. 17C). None of these capabilities existing with current system or methods.

Continuing in the user insights module, Kira is able to see new and enriched statistics about her system which is not currently represented in any existing systems or methods. She is able to see progress on how many of her states (parts) she has checked in with that have requested a check-in (see FIG. 11A), how many check-ins total she has completed, her newly added states (parts), and her top 3 most active states (parts) over a given period of time (see FIG. 17A). This represents a wonderful step forward with new and enriched experiences as there is currently no way to obtain these types of statistics on any easy basis. These statistics are monumentally helpful for Kira in her journey of self-transformation as she will be able to head into her therapy appointments prepared to utilize her time well having specific knowledge about what she would like to work with and what has been a struggle for her recently. The representation of the number of check-ins being in a circle (see FIG. 17A) showing how close Kira is to reaching her goal is inspiring and encouraging to her. The inspiration and insight she gets from her insights in the tool 101 removes barriers to change such as frustration, boredom, confusion and lack of knowledge. With these insights she feels empowered and less overwhelmed as she continues on her journey. The same statistics can be found for trailheads.

FIG. 5 depicts the user insights 150 core module of the awareness and support tool 101. user insights comprises states 192, triggers 193, and Overall Activity 151. States 192 comprises check-in loop 192E, New 192F, and Most active 192G. Triggers 193 comprises check-in loop 193D, New 193E, and Most active 193F. Overall Activity 151 comprises Visual Representation Across Time 151A and Total Time Breakdown 151B.

Check-in Loops 192E and 193D gathers information from the user 102 as to the defined number of times a user 102 has selected to check in with the respective state or trigger, and visually represents how close a user is to the defined goal of checking in with the respective state or trigger for the defined period of time. See FIG. 17A.

New 192F module and New 193E modules define new respective states and triggers and help a user 102 to clarify and organize thoughts when headed into a therapy session or for their own self-reflection. Seeing which states or triggers are new helps reduce the amount of time “checking in” at the beginning of a therapy session

Most active 192G and Most active 193F provide a list of respective most active respective states and triggers and help a user to clarify and organize thoughts when headed into a therapy session or for their own self-reflection. Generally speaking, much time is spent at the beginning of a therapy session updating the therapist of what has gone on recently in the user's life. However, with the list of most recent or most active states or triggers a user is able to quickly and clearly identify areas that need the most attention and better utilize their time with their professional.

Visual Representation Across Time 151A provides a visual representation of overall activity in the tool 101 and shows the user how much time they have been spending connecting with their internal experience over the variety of different areas within the tool 101. It can help the user to see that the more time they spend in the tool can correlate with increased levels of access to core self and vice versa. If a user hasn't been spending a lot of time with their internal experience and they see poor core self-assessment scores it may motivate them to get back to engaging the tool 101. See FIG. 17B.

Attorney No. 2028-2

Total Time Breakdown 151B shows the user where in the tool 101 they spend their time while using the tool to give them insights to see what of the nine integration tools and what of the eight core modules are most helpful to their journey of self-transformation. See FIG. 11C.

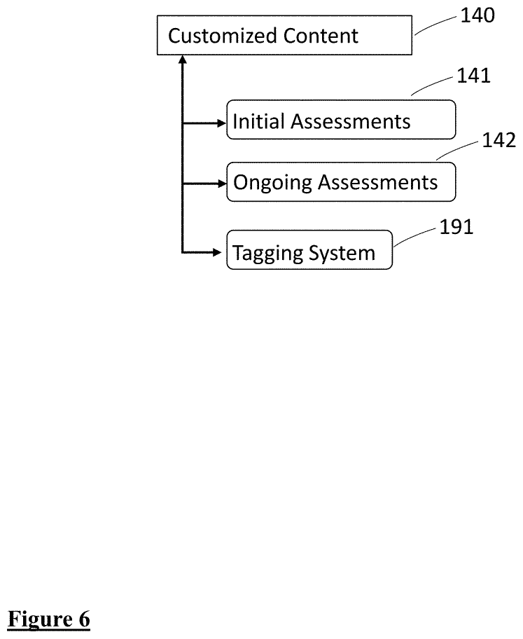

As depicted in FIG. 6, the Customized Content 140 core module of the tool 101 comprises initial assessments 141, ongoing assessments 142, and tagging system 191.

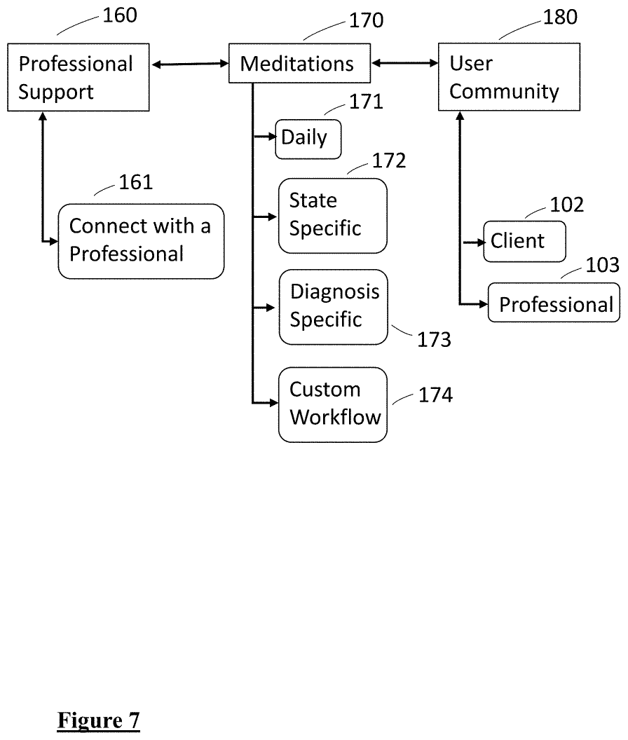

FIG. 7 depicts each of the Professional Support 160, Guided exercises 170, and User Community 180 core modules. The professional Support 160 module comprises and interacts with a Connect with a Professional 161 element, The Guided exercises 170 module comprises and interacts with a Daily 171, state Specific 172, Diagnosis Specific 173, and Custom Workflow 174 modules. The User Community 180 module comprises and interacts with Client 102 and Professional (e.g., Therapist).

With attention to FIGS. 16A-D, the core self 194 may be monitored, tracked, and evaluated. Returning yet again to the Kira use case, Kira utilizes the core self (Self) button from the home page (see FIG. 8A, element 804) which takes her to a visual representation of her perceived average level of access to her core self (Self) (see FIG. 16A). From here she goes to her assessment breakdown (see FIG. 16B) to determine if the recent assessment is showing an increase or decrease in her average level of specific qualities by the tick mark in the horizontal bar. This gives her incredible insight into her overall self-transformation journey. She can see insights like how certain qualities relate to each other (see FIG. 16B). She also can easily check her perceived level of access to core self (Self) (see FIG. 16D) and set reminders to check this access with herself often. The visual representation, assessments and reminders are wonderful tools for her as right now she is currently just doing a mental check in on how she is feeling when she thinks of it. This is not very helpful to her when she is feeling overwhelmed or hopeless. Those tough days can feel like they are every day with doing it that way, whereas, using the Self portion of the tool 101 she is able to see such progress in her journey and it continues to encourage her through the tough days.

A number of variations and modifications of the disclosure can be used. It would be possible to provide for some features of the disclosure without providing others.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An awareness and support tool to improve user well-being of a user, comprising:
a user interface operating to receive temporally-marked user data sets comprising:
i) user state data comprising user mental state and user physical state; ii) user trigger data;
iii) user self-assessment data; and iv) user self-reflection data;
a user database operating to: i) store the temporally-marked user data sets; ii) sort the temporally-marked user data sets; and iii) filter the temporally-marked user data sets;
a system database operating to store a set of guided exercises associated with at least user state data, user trigger data, and user self-assessment data;
a processor operating on the user database to determine mappings between data of the temporally-marked user data sets and to identify insights associated with data of the temporally-marked user data sets;
wherein:
the user inputs, through the user interface, a first set of user self-assessment data and a first set of user state data;
the processor selects, based on the first set of user self-assessment data and the first set of user state data, a first guided exercise from the system database, and renders the first guided exercise on the user interface;
the user executes the guided exercise and inputs, through the user interface, a first set of user self-reflection data associated with executing the first guided exercise;
the user inputs, through the user interface, a first set of user trigger data;
the processor maps the first set of user state data and the first set of user trigger data with respect to a set of pre-existing temporally-marked user data sets to create a first visual map;
the processor renders, on the user interface, the first visual map;
the processor generates a first insight associated with the first set of user state data, the first set of user trigger data, and the set of pre-existing temporally-marked user data;
the processor renders the first insight; and
the user inputs, through the user interface, a second set of user self-assessment data.

2. The awareness and support tool of claim 1, wherein the processor compares the first set of user self-assessment data and the second set of user self-assessment data to generate a second insight, the second insight render on the user interface by the processor.

3. The awareness and support tool of claim 1, wherein the first set of user self-assessment data comprises a visual sliding scale data entry.

4. The awareness and support tool of claim 1, wherein the first visual map comprises a particular set of user state data for a selected user trigger data.

5. The awareness and support tool of claim 4, wherein the set of user state data is rendered as a first particular set of user state data and a second particular set of user data.

6. The awareness and support tool of claim 1, wherein the user enters a time reminder to view a selected trigger.

7. The awareness and support tool of claim 1, wherein temporally-marked user data sets further comprise user parts data.

8. The awareness and support tool of claim 7, wherein the processor renders, on the user interface, a second visual map comprising a particular set of user state data for a selected user parts data.

9. The awareness and support tool of claim 1, wherein the processor and user interface reside on a portable computer device.

10. The awareness and support tool of claim 9, wherein at least part of the user database and the system database reside external to the portable computer device.

11. An awareness and support tool to improve user well-being of a user, comprising:
a user database operating to i) store a plurality of user part data sets, each user part data set comprising a set of current user state data and a set of existing user state data; ii) sort the plurality of user part data sets with respect to a particular set of user state data; and
iii) filter the plurality of user part data sets with respect to the particular set of user state data;
a user interface operating to receive: i) the set of current user state data, the current user state data comprising user mental state and user physical state; and ii) user self-assessment data;
a system database operating to store a set of guided exercises associated with at least the current user state data;
a processor operating on the user database to determine mappings between the plurality of user part data sets and at least one of the set of current user state data and the set of existing user state data;
wherein:
the processor generates and renders a first visual mapping of a relationship between the plurality of user part data sets and at least one of the set of current user state data and the set of existing user state data;
the user selects the particular set of user state data as rendered on the user interface by the processor;
the user enters the set of current user state data associated with the particular set of user state data; and
the processor selects, based on the set of current user state data, a first guided exercise from the system database, and renders the first guided exercise on the user interface.

12. The awareness and support tool of claim 11, wherein the user executes the guided exercise and inputs, through the user interface, a first set of user self-reflection data associated with executing the first guided exercise.

13. The awareness and support tool of claim 12, wherein the first set of user self-assessment data comprises a visual sliding scale data entry.

14. The awareness and support tool of claim 11, wherein the processor generates a first insight associated with the set of current user state data.

15. The awareness and support tool of claim 11, wherein the user inputs, through the user interface, i) a first set of user self-assessment data before the user selects the particular set of user state data, and ii) a second set of user self-assessment data after the user executes the first guided exercise.

16. A method of improving user well-being of a user, comprising:
providing an awareness and support tool comprising:
to a user interface operating to receive temporally-marked user data sets comprising: i) user state data comprising user mental state and user physical state; ii) user trigger data; iii) user self-assessment data; and iv) user self-reflection data;
a user database operating to: i) store the temporally-marked user data sets;
ii) sort the temporally-marked user data sets; and iii) filter the temporally-marked user data sets;
a system database operating to store a set of guided exercises associated with at least user state data, user trigger data, and user self-assessment data; and
a processor operating on the user database to determine mappings between data of the temporally-marked user data sets and to identify insights associated with data of the temporally-marked user data sets;
inputting, by the user through the under interface, a first set of user self-assessment data and a first set of user state data;
selecting, by the processor and based on the first set of user self-assessment data and the first set of user state data, a first guided exercise from the system database;
rendering, by the processor, the first guided exercise on the user interface;
executing, by the user, the guided exercise;
inputting, by the user through the user interface, a first set of user self-reflection data associated with executing the first guided exercise;
inputting, through the user interface, a first set of user trigger data;
mapping, by the processor, the first set of user state data and the first set of user trigger data with respect to a set of pre-existing temporally-marked user data sets to create a first visual map;
rendering, by the processor on the user interface, the first visual map;
generating, by the processor, a first insight associated with the first set of user state data, the first set of user trigger data, and the set of pre-existing temporally-marked user data;
rendering, by the processor, the first insight; and
inputting, by the user through the user interface, a second set of user self-assessment data.

17. The method of claim 16, wherein the first set of user self-assessment data comprises a visual sliding scale data entry.

18. The method of claim 16, wherein the first visual map comprises a particular set of user state data for a selected user trigger data.

19. The method of claim 16, wherein the set of user state data is rendered as a first particular set of user state data and a second particular set of user data.

20. The method of claim 16, wherein: i) the processor and user interface reside on a portable computer device; and ii) at least part of the user database and the system database reside external to the portable computer device.
